(19) United States
(12) Patent Application Publication
(10) Pub. No.: US 2026/0123849 A1
(43) Pub. Date: May 7, 2026

(54) DISTORTION MODELING AND COMPENSATION IN A CURVE-TRACKED DETECTOR ARRAY

(71) Applicant: Magnisity Ltd., Hod-HaSharon (IL)

(72) Inventors: Ron BARAK, Tel-Aviv (IL); Benjamin GREENBURG, Hod-HaSharon (IL); Amit COHEN, Ramat Gan (IL); Eyal KLEIN, Tel-Aviv (IL)

(73) Assignee: Magnisity Ltd., Hod-HaSharon (IL)

(21) Appl. No.: 19/117,987

(22) PCT Filed: Oct. 3, 2023
(86) PCT No.: PCT/IL2023/051059

§ 371 Date: Apr. 3, 2025

Related U.S. Application Data
(60) Provisional application No. 63/412,559, filed on Oct. 3, 2022.
(60) Provisional application No. 63/536,467, filed on Sep. 4, 2023.

(51) Int. Cl. A61B 5/06; A61B 34/20

(52) U.S. Cl. CPC A61B 5/065; A61B 34/20; A61B 2034/2051; A61B 2034/2061

(57) ABSTRACT

A system for position and/or shape-tracking of an elongated device including a shape sensor to estimate a position and orientation of the device at a plurality of points along the device, and a processor configured to: obtain a plurality of predetermined points along a tracked portion of the device; allocate, for each of the plurality of the predetermined points, a local energy function dependent on the estimated position and orientation of device that incorporates relevant mechanical and sensor measurement constraints; generate a resultant unified energy function for the full shape and position of the entire tracked portion of the device with respect to relative locations and orientation of adjacent plurality of the predetermined points, the unified energy function is constructed based on the allocated local energy functions and segmental energy functions; and calculate a fully localized curve along the tracked portion of the device by minimizing the energy function.

### RELATED APPLICATION/S

This application claims the benefit of priority of U.S. Provisional Patent Application No. 63/412,559 filed on 3 Oct. 2022 and further claims the benefit of priority of U.S. Provisional Patent Application No. 63/536,467 filed on 4 Sep. 2023, the contents of which are incorporated herein by reference in their entirety.

### FIELD AND BACKGROUND OF THE INVENTION

The present invention, in some embodiments thereof, relates to system and methods for position and/or shape-tracking of an elongated device and, more particularly, but not exclusively, to system and methods for performing corrections on position and/or shape-tracking of an elongated device, optionally taking under consideration electromagnetic distortions.

Electromagnetic tracking systems are widely used in clinical applications to track certain instruments inside the patient's body in 3D. A common electromagnetic tracking system usually consists of an electromagnetic transmitter, which generates a number of different alternating electromagnetic fields, commonly at different frequencies (for example, 3 different fields at frequencies 1 Khz, 2 Khz, 3 Khz) and an electromagnetic sensor which usually consists of one or more electromagnetic coils (for example, 3 concentric small electromagnetic coils). The alternating fields generate Electromotive Force (EMF) in the sensor's coils which are sensed on the receiving end. The measured fields are then used to compute the position and orientation of the electromagnetic sensor. The solution of a 6 Degrees-of-Freedom (6-DOF) or 5-DOF or any other configuration of solved position and orientation of the sensor relative to the transmitter relies on the knowledge of the values of the generated EM fields at each point in space relative to the transmitter. By knowing the values of the generated fields, the receiver is able to determine the position and orientation of the sensor in space relative to the transmitter such that the measured fields correspond to its solved position and orientation.

Certain objects are known to create an electromagnetic distortion in space and impact the accuracy of the solved position and orientation relative to the transmitter. For example, certain ferromagnetic/paramagnetic/diamagnetic materials (collectively, magnetic materials) may be magnetized due to the electromagnetic fields generated by the transmitter and become sources for electromagnetic fields (of similar frequencies). Conductive materials may serve as receivers in the sense that they experience EMF due to the generated electromagnetic fields. These EMF create electrical currents (eddy currents) inside the conductive metals which generate secondary fields, such that the conductive metals may also become sources for electromagnetic fields (of similar frequencies) on their own. In a static environment, where all magnetic and conductive materials are positioned and oriented statically relative to the transmitter and/or the receiver, the distortion fields can be modeled and learned in a mapping and calibration process prior to operating the tracking system. For example, in a clinical environment, where a transmitter is fixed to a patient's bed, conductive metals on the bed are located in a static position relative to the transmitter. In this case, the distortion fields which are caused by eddy currents flowing through the conductive metals or by magnetization of magnetic metals are static in the sense that they do not change during operation of the tracking system. By mapping the total field generated in the sensing volume surrounding the transmitter, the system can then use the mapped fields (rather than the “neutral” or theoretically expected fields) to perform the tracking. In another example, a magnetic stainless-steel metal is located inside an endoscope, having a magnetic sensor (EM sensor). The magnetic metal creates a distortion field as described above. Since the magnetic sensor is fixed to the distorter, the distortion field moves together with the magnetic sensor and its effect is static and can be modeled in a calibration process. For example, the distortion field's effect may be modeled as increased sensing gain of the EM sensor, or more generally, as a gain matrix applied to measurements of three concentric sensing coils, regardless of the sensor's position and orientation in space.

Additional background art includes U.S. Pat. No. 11,712,309 disclosing an EM shape sensor which consists of a sensor-array made of multiple discrete digital 3D magnetometers assembled on a Flexible Printed Circuit (FPC). The sensor-array may be embedded in an endoscope (or other tubular device) to enable EM shape sensing of that endoscope.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A system for position and/or curve/shape-tracking of an elongated device, comprising:
a. a curve/shape sensor comprising a plurality of sensor elements positioned on said elongated device;
b. one or more transmitters;
c. a controller comprising a processor; said processor comprising instructions for:
   i. obtaining a plurality of points along a tracked portion of said elongated device;
   ii. allocating, for each point from said plurality of points, a local energy function dependent on an estimated position and orientation of said tracked portion of said elongated device;
   iii. generating a resultant unified energy function for a full shape and a position of an entire tracked portion of said elongated device;
   iv. calculating a fully localized curve along said tracked portion of said elongated device by minimizing said unified energy function.

Example 2. The system according to example 1, wherein said local energy function incorporates relevant mechanical and sensor measurement constraints for said each point.

Example 3. The system according to example 2, wherein said unified energy function is constructed based on said allocated local energy functions and segmental energy functions that relate to said constraints of mechanical properties of said elongated device, with respect to relative locations and orientation of adjacent plurality of the predetermined points.

Example 4. The system according to any one of examples 1-3, wherein said fully localized curve is calculated relative to said one or more transmitters.

Example 5. The system according to any one of examples 1-4, wherein said plurality of points are a predetermined plurality of points.

Example 6. The system according to any one of examples 1-5, wherein said plurality of points are one or more of:
a. a sensor point, which is a point in which said sensor elements of said shape sensor are located; and
b. a curve point, which is a virtual predetermined point positioned at predetermined intervals between sensor points.

Example 7. The system according to example 6, wherein there are a plurality of sensor points along said tracked portion of said elongated device.

Example 8. The system according to example 6, wherein there are one or more curve points between sensor points.

Example 9. The system according to any one of examples 1-8, wherein said local energy function incorporates constraints related to a sensed magnetic field.

Example 10. The system according to any one of examples 1-9, wherein the processor further comprises instructions for allocating a weight for each local energy function, based on a certainty value, related to a certainty that a measurement is accurate.

Example 11. The system according to example 3, wherein said segmental energy function is a length energy function, related to known distances along said elongated device between adjacent points from said plurality of points or a known length of said tracked portion of said elongated device.

Example 12. The system according to example 11, wherein said length energy function incorporates an approximation of a total curve length between two points, and a known distance along said elongated device between said two points.

Example 13. The system according to example 11, wherein said length energy function relates to a length along said elongated device between two adjacent curve points, and is proportional to the squared difference between a known distance and a linearly approximated distance according to momentarily calculated positions.

Example 14. The system according to example 3, wherein said segmental energy function is an orientation energy function, related to a limited possible orientation difference between adjacent points from said plurality of points.

Example 15. The system according to example 14, wherein said orientation energy function grows in accordance with said orientation difference between said adjacent points.

Example 16. The system according to example 3, wherein said segmental energy function is at least one selected from the group consisting of:
a. at least one energy function corresponding to the tracking approximation of a sensor point;
b. at least one energy function corresponding to the length approximation between adjacent points;
c. at least one energy function corresponding to the distortion approximation of a sensor point;
d. at least one energy function corresponding to the orientation difference between adjacent points;
e. at least one energy function corresponds to the twist difference between adjacent points;
f. at least one energy function corresponds to the smoothness/curvature difference between adjacent points; and
g. wherein at least one energy function corresponds to the motion difference between a point and a motion model of that point.

Example 17. The system according to example 3, wherein said segmental energy function is a smoothness energy function configured for minimizing a curvature along a sequence of adjacent points.

Example 18. The system according to example 3, wherein said segmental energy function is a motion energy function configured for minimizing a jitter of said calculated fully localized curve.

Example 19. The system according to any one of examples 1-18, wherein said processor further comprises instructions for calibrating said curve/shape sensor with respect to a magnetic field distortion imposed by a tool inserted into said elongated device.

Example 20. The system according to example 19, wherein said calibration includes one or more of:
a. obtaining a plurality of distortion samples;
b. finding distortion calibration parameters by reducing a distortion matrix based on said distortion samples; and
c. using said distortion calibration parameters to adjust said calculated fully localized curve of said tracked portion of said elongated device.

Example 21. The system according to example 20, wherein said calibration further comprises monitoring a location of a tool tip.

Example 22. The system according to example 21, wherein said monitoring comprises comparing and fitting a theoretical diagram of a distortion distribution to a series of distortion values calculated for a plurality of points along said tracked portion of said elongated device.

Example 23. The system according to example 21, wherein said distortion value for a certain sensor element is calculated by a rooted mean square of said reduced distortion matrix.

Example 24. The system according to any one of examples 1-23, wherein said one or more transmitters are configured for transmitting one or more multi-frequency EM fields comprising one or more harmonies of a base frequency.

Example 25. The system according to example 24, wherein said processor comprises further instructions for:
a. analyzing sensed magnetic fields;
b. comparing said sensed magnetic fields with an expected frequency profile;
c. extracting a distortion field; and
d. utilizing said distortion field in said calculating.

Example 26. A computer implemented method for position and/or curve/shape-tracking of an elongated device performed by a curve/shape sensor comprising a plurality of sensor elements positioned on said elongated device, the method comprising:
a. obtaining a plurality of points along a tracked portion of said elongated device; said plurality of points corresponding to readings from said plurality of sensor elements;
b. allocating, for each point from said plurality of points, a local energy function dependent on an estimated position and orientation of said tracked portion of said elongated device;
c. generating a resultant unified energy function for a full shape and a position of an entire tracked portion of said elongated device;
d. calculating a fully localized curve along said tracked portion of said elongated device by minimizing said unified energy function.

Example 27. The computer implemented method according to example 26, wherein said local energy function incorporates relevant mechanical and sensor measurement constraints for said each point.

Example 28. The computer implemented method according to example 27, wherein said unified energy function is constructed based on said allocated local energy functions and segmental energy functions that relate to said constraints of mechanical properties of said elongated device, with respect to relative locations and orientation of adjacent plurality of the predetermined points.

Example 29. The computer implemented method according to any one of examples 26-28, wherein said fully localized curve is calculated relative to one or more transmitters.

Example 30. The computer implemented method according to any one of examples 26-29, wherein said plurality of points are a predetermined plurality of points.

Example 31. The computer implemented method according to any one of examples 26-30, wherein said plurality of points are one or more of:
a. a sensor point, which is a point in which said sensor elements of said shape sensor are located; and
b. a curve point, which is a virtual predetermined point positioned at predetermined intervals between sensor points.

Example 32. The computer implemented method according to example 31, wherein there are a plurality of sensor points along said tracked portion of said elongated device.

Example 33. The computer implemented method according to example 31, wherein there are one or more curve points between sensor points.

Example 34. The computer implemented method according to any one of examples 26-33, wherein said local energy function incorporates constraints related to a sensed magnetic field.

Example 35. The computer implemented method according to any one of examples 26-34, further comprising allocating a weight for each local energy function, based on a certainty value, related to a certainty that a measurement is accurate.

Example 36. The computer implemented method according to example 28, wherein said segmental energy function is a length energy function, related to known distances along said elongated device between adjacent points from said plurality of points or a known length of said tracked portion of said elongated device.

Example 37. The computer implemented method according to example 36, wherein said length energy function incorporates a linear approximation of a total curve length between two points, and a known distance along said elongated device between said two points.

Example 38. The computer implemented method according to example 36, wherein said length energy function relates to a length along said elongated device between two adjacent curve points, and is proportional to the squared difference between a known distance and a linearly approximated distance according to momentarily calculated positions.

Example 39. The computer implemented method according to example 28, wherein said segmental energy function is an orientation energy function, related to a limited possible orientation difference between adjacent points from said plurality of points.

Example 40. The computer implemented method according to example 28, wherein said segmental energy function is at least one selected from the group consisting of:
a. at least one energy function corresponding to the tracking approximation of a sensor point;
b. at least one energy function corresponding to the length approximation between adjacent points;
c. at least one energy function corresponding to the distortion approximation of a sensor point;
d. at least one energy function corresponding to the orientation difference between adjacent points;
e. at least one energy function corresponds to the twist difference between adjacent points;
f. at least one energy function corresponds to the smoothness/curvature difference between adjacent points; and
g. wherein at least one energy function corresponds to the motion difference between a point and a motion model of that point.

Example 41. The computer implemented method according to example 39, wherein said orientation energy function grows in accordance with said orientation difference between said adjacent points.

Example 42. The computer implemented method according to example 28, wherein said segmental energy function is a smoothness energy function configured for minimizing a curvature along a sequence of adjacent points.

Example 43. The computer implemented method according to example 28, wherein said segmental energy function is a motion energy function configured for minimizing a jitter of said calculated fully localized curve.

Example 44. The computer implemented method according to any one of examples 26-43, further comprising calibrating said curve/shape sensor with respect to a magnetic field distortion imposed by a tool inserted into said elongated device.

Example 45. The computer implemented method according to example 44, wherein said calibrating includes one or more of:
a. obtaining a plurality of distortion samples;
b. finding distortion calibration parameters by reducing a distortion matrix based on said distortion samples; and
c. using said distortion calibration parameters to adjust said calculated fully localized curve of said tracked portion of said elongated device.

Example 46. The computer implemented method according to example 44, wherein said calibration further comprises monitoring a location of a tool tip.

Example 47. The computer implemented method according to example 46, wherein said monitoring comprises comparing and fitting a theoretical diagram of a distortion distribution to a series of distortion values calculated for a plurality of points along said tracked portion of said elongated device.

Example 48. The computer implemented method according to example 45, wherein said distortion value for a certain sensor element is calculated by a rooted mean square of said reduced distortion matrix.

Example 49. A non-transitory computer-readable storage medium having stored thereon executable instructions that, as a result of being executed by a processor of a computer system, cause the computer system to at least perform a computer-implemented method of example 26.

Example 50. A system for position and/or shape-tracking of an elongated device, the system comprises:
a. a shape sensor to estimate a position and orientation of the device at a plurality of points along the device; and
b. a processor configured to:
   i. obtain a plurality of predetermined points along a tracked portion of the device;
   ii. allocate, for a plurality of the predetermined points, a local energy function dependent on the estimated position and orientation of device at this point, that incorporates relevant mechanical and sensor measurement constraints for the point;
   iii. generate a resultant unified energy function for the full shape and position of the entire tracked portion of the device, the unified energy function is constructed based on the allocated local energy functions and segmental energy functions that relate to constraints of mechanical properties of the device, with respect to relative locations and orientation of adjacent plurality of the predetermined points;
   iii. calculate a fully localized curve along the tracked portion of the device by minimizing the energy function.

Example 51. The system according to example 50, wherein the predetermined points include sensor points, which are points in which sensor elements of the shape sensor are located, and curve points, which are virtual predetermined number of points in predetermined intervals between the sensor points.

Example 52. The system according to example 50 or example 51, wherein the energy function for a certain point along the device may incorporate constraints related to the sensed magnetic field at this point.

Example 53. The system according to any one of examples 50-52, wherein the processor further allocates a weight for each local energy function, based on a certainty value, related to a certainty that a measurement is accurate.

Example 54. The system according to any one of examples 50-53, wherein a segmental energy function is a length energy function, related to known distances along the device between adjacent predetermined points or a known length of a tracked portion of device.

Example 55. The system according to example 54, wherein the length energy function incorporates a linear approximation of the total curve length between two points, and a known distance along the device between the two points.

Example 56. The system according to example 54, wherein the length energy function relates to the length along the device between two adjacent curve points, and is proportional to the squared difference between a known distance and a linearly approximated distance according to momentarily calculated positions.

Example 57. The system according to any one of examples 50-56, wherein a segmental energy function is an orientation energy function, related to a limited possible orientation difference between adjacent points.

Example 58. The system according to example 57, wherein the orientation energy function grows in accordance with the orientation difference between adjacent points.

Example 59. The system according to any one of examples 50-58, wherein a segmental energy function is a smoothness energy function, striving to minimize a curvature along a sequence of adjacent points.

Example 60. The system according to any one of examples 50-59, wherein the processor is further configured to calibrate the shape sensor with respect to a magnetic field distortion imposed by tool inserted into the device.

Example 61. The system according to example 60, wherein the calibration includes:
a. obtaining a plurality of distortion samples;
b. finding distortion calibration parameters by reducing a distortion matrix based on the distortion samples; and
c. use distortion calibration parameters to adjust the solved curve and position of tracked device.

Example 62. The system according to example 61, further comprising monitoring the location of a tool tip, the monitoring comprises comparing and fitting a theoretical diagram of the distortion distribution to a series of distortion values calculated for a plurality of points along the device.

Example 63. The system according to example 62, wherein the distortion value for a certain sensor element is calculated by a rooted mean square of the reduced distortion matrix.

According to an aspect of some embodiments of the present invention there is provided a system for position and shape-tracking of an elongated device, the system comprises: a shape sensor (The term “shape sensor” refers hereinafter to any one or more sensors configured to be used for the required tasks, for example to calculate and/or track a position and/or a shape. The term “shape sensor” is also referred to as a position sensor or as “sensor array”, or as “sensor grid”, or as a curve sensor, or as curve/shape sensor, or EM sensor) to estimate a position and orientation of the device at a plurality of points along the device; a processor configured to: obtain a plurality of predetermined points along a tracked portion of the device; allocate, for a plurality of the predetermined points, a local energy function dependent on the estimated position and orientation of device at this point, that incorporates relevant mechanical and sensor measurement constraints for the point; generate a resultant unified energy function for the full shape and position of the entire tracked portion of the device, the unified energy function is constructed based on the allocated local energy functions and segmental energy functions that relate to constraints of mechanical properties of the device, with respect to relative locations and orientation of adjacent plurality of the predetermined points; calculate a fully localized curve along the tracked portion of the device by minimizing the energy function.

According to some embodiments of the invention, the predetermined points include sensor points, which are points in which sensor elements of the shape sensor are located, and curve points, which are virtual predetermined number of points in predetermined intervals between the sensor points.

According to some embodiments of the invention, the energy function for a certain point along the device incorporates constraints related to the sensed magnetic field at this point.

According to some embodiments of the invention, the processor further allocates a weight for each local energy function, based on a certainty value, related to a certainty that a measurement is accurate.

According to some embodiments of the invention, a segmental energy function is a length energy function, related to known distances along the device between adjacent predetermined points or a known length of a tracked portion of device.

According to some embodiments of the invention, the length energy function incorporates an approximation (for example, a linear approximation) of the total curve length between two points, and a known distance along the device between the two points.

According to some embodiments of the invention, the length energy function relates to the length along the device between two adjacent curve points, and is proportional to the squared difference between a known distance and an approximated distance according to momentarily calculated positions.

According to some embodiments of the invention, a segmental energy function is an orientation energy function, related to a limited possible orientation difference between adjacent points.

According to some embodiments of the invention, the orientation difference is computed as the orientation of a point i relative to the orientation of its preceding point i−1, for example,

$${R}_{i - 1}^{T} {R}_{i}$$

(the orientation of point i in point i−1 coordinate system), or as a full relative transform (which also includes relative position)

$${T}_{i - 1}^{- 1} {T}_{i} .$$

According to some embodiments of the invention, the orientation function grows in accordance with the orientation difference between adjacent points, for example, in accordance with:

$${‖ {R}_{i - 1}^{T} {R}_{i} - {I}_{3} ‖}^{2} .$$

This can also be written in quaternion form:

$${‖ {q}_{i - 1}^{- 1} {q}_{i} - ( 1 , 0 , 0 , 0 ) ‖}^{2} ,$$

or can also account for relative position:

$${‖ {T}_{i - 1}^{- 1} {T}_{i} - {T}_{i , i - 1}^{0} ‖}^{2} where {T}_{i , i - 1}^{0}$$

is the initial (calibrated) relative transform of point i relative to point i−1.

According to some embodiments of the invention, the orientation energy function grows very rapidly (for example, polynomially or exponentially) when the orientation difference between adjacent points is larger than a threshold value. In some embodiments, an exemplary threshold value is characterized by being a “soft” value provided specifically for a certain situation.

According to some embodiments of the invention, a segmental energy function is a smoothness energy function, striving to minimize the curvature along a sequence of adjacent points.

According to some embodiments of the invention, a segmental energy function is a 2^{nd} order smoothness energy function, striving to minimize the change in curvature along a sequence of adjacent points (see below).

According to some embodiments of the invention, a temporal energy function is used to incorporate temporal sensor data, striving to minimize the deviation between the solved sensor's motion over time and an applied motion model (see below).

According to some embodiments of the invention, by using an energy function which incorporates numerous (or at least one) constraints to solve for the sensor's shape, the solved shape is not only more immune to electromagnetic distortion, but also contains less noise (due to magnetic measurement noise) such that the solved shape is less jittery over time. In some embodiments, this is achieved because the solved shape is constrained (for example, deliberately configured) to mechanically plausible solutions, which reduces the degrees of freedom of the solution. It may also be achieved by combining a motion energy function.

According to some embodiments of the invention, the sensor array comprises points which are connected in a general graph connectivity rather than being necessarily connected in a sequential manner, for example, being embedded in a tracked glove. In some embodiments, the sensor array is then used to track the graph structure of a device rather than just a curve, using the methods described herein.

According to some embodiments of the invention, the energy functions described herein can be applied on a graph connectivity rather than on sequentially connected points to track the graph structure of a device rather than just a curve.

According to some embodiments of the invention, the processor is further configured to calibrate the shape sensor with respect to a magnetic field distortion imposed by tool inserted into the device.

According to some embodiments of the invention, the calibration includes: obtaining a plurality of distortion samples; finding distortion calibration parameters by, for example, reducing a distortion matrix based on the distortion samples; and use distortion calibration parameters to adjust the solved curve and position of tracked device.

According to some embodiments of the invention, the system further comprising monitoring the location of a tool tip, the monitoring comprises comparing and fitting a theoretical diagram of the distortion distribution to a series of distortion values calculated for a plurality of points along the device.

According to some embodiments of the invention, the distortion value for a certain sensor element is calculated by a rooted mean square of the reduced distortion matrix.

According to an aspect of some embodiments of the present invention there is provided a shape sensor which consists of a plurality of sensor elements and a curve which is solved and fitted between the sensor elements under imposed constraints.

According to some embodiments of the invention, a smoothness constraint is imposed on the solved curve.

According to some embodiments of the invention, a length constraint is imposed on the solved curve.

According to some embodiments of the invention, the curve is fitted between individually solved positions and orientations.

According to some embodiments of the invention, the curve is directly fitted using the measured magnetic fields of each sensor element.

According to some embodiments of the invention, a distortion model is further solved and distortion fields are subtracted in the curve fitting process.

According to some embodiments of the invention, the distortion model describes the distortion fields of a magnetic tool (The term “magnetic tool” refers herein, and throughout the present disclosure, to any object capable of generating a magnetic distortion to a magnetic field, for example in an operational environment, such as a mechanical arm of an imaging system, a bed frame, surgical tools, etc.). In some embodiments, the magnetic distortion can be caused due to magnetic metals and/or due to eddy currents of conductive materials and/or due to any other cause of magnetic distortion.

According to some embodiments of the invention, the distortion model describes the distortion fields of a global distorter, such as a C-arm.

According to some embodiments of the invention, the shape sensor is an EM shape sensor and the sensor elements are EM sensor elements.

According to some embodiments of the invention, the EM sensor elements are digital magnetometers.

According to some embodiments of the invention, the shape sensor consists of an FPC.

According to some embodiments of the invention, the FPC is integrated inside an endoscope.

According to some embodiments of the invention, the FPC is wrapped around an endoscope's working channel.

According to an aspect of some embodiments of the present invention there is provided detection of distortion of sensor elements along a shape sensor by a system.

According to some embodiments of the invention, the distortion detection is used to determine the presence of a distorter, such as a magnetic tool, along the shape sensor's length.

According to some embodiments of the invention, the distortion detection is used to detect the type of distorter, such as the tool which was introduced to a tracked endoscope's working channel.

According to some embodiments of the invention, the system prompts the user with the automatically detected tool for confirmation.

According to some embodiments of the invention, the system asks the user to choose a tool from a list of available tools.

According to some embodiments of the invention, the tool's distortion model is calibrated in advance.

According to some embodiments of the invention, the distortion detection is used to determine the position of a magnetic tool along the shape sensor's length.

According to some embodiments of the invention, the distortion detection is used to determine the position of a magnetic clinical tool (also referred as surgical tool, working channel tool) inside a shape tracked endoscope.

According to some embodiments of the invention, the tool is displayed in its tracked position in 3D.

According to some embodiments of the invention, the distortion detection is used to decrease the tracking weight of one or more sensor elements of the sensor array to improve the accuracy of the final solved curve.

According to some embodiments of the invention, at least one sensor is fitted to a tool to aid in the detection, modeling and compensation of the tool distortion.

According to some embodiments of the invention, data from a drive system is used to aid in the detection, modeling, and compensation of a driven distorter, such as a robotic arm.

According to an aspect of some embodiments of the present invention there is provided a general energy-based framework for simultaneously solving the position and orientation of a plurality of sensors.

According to some embodiments of the invention, global distortion information is shared using the same distortion model and parameters between all solved sensors.

According to some embodiments of the invention, all sensors experience the global distortion caused by same global distorter and share the distortion model information.

According to some embodiments of the invention, global distortion is jointly solved by the simultaneous solving of all tracked sensors in a single framework.

According to some embodiments of the invention, a sensor grid is used to enhance the detection and compensation of global distortion, such as a C-arm's.

According to some embodiments of the invention, the sensor grid is solved with additional structural constraints.

According to some embodiments of the invention, the sensor grid is attached to a patient's chest.

According to some embodiments of the invention, the sensor grid is attached to the patient's head.

According to some embodiments of the invention, the sensor grid is used to track patient's motion.

According to some embodiments of the invention, the sensor grid is used to track patient's breathing.

According to some embodiments of the invention, the sensor grid is used to model the deformation of a patient's internal organ by tracking the patient's body movement and deformation.

According to some embodiments of the invention, multiple distortion models and fields can be described in the same energy minimization framework.

According to some embodiments of the invention, each individually position and/or shape-tracked device can have its own tool distortion model and fields.

According to some embodiments of the invention, all devices can share the same global distortion model and fields.

According to some embodiments of the invention, the mutual distortion of multiple devices or tools is detected.

According to some embodiments of the invention, the mutual distortion of multiple devices or tools is compensated.

According to an aspect of some embodiments of the present invention there is provided a low-frequency EM tracking system (position and/or shape-tracking system) with reduced distortion.

According to some embodiments of the invention, the low-frequency EM tracking system comprises sensor elements which are DC magnetometers, and their SNR is unaffected by the low-frequency fields in use, while distortion is reduced.

According to some embodiments of the invention, the low-frequency EM tracking system comprises a transmitter that generates low-frequency EM fields which decreases distortion due to eddy currents in nearby conducting distorters.

According to some embodiments of the invention, the transmitter generates EM fields of a special multi-frequency profile.

According to some embodiments of the invention, the transmitter generates EM fields of low base frequency (for example, lower than 300 Hz)

According to some embodiments of the invention, the transmitter generates rectangular, triangular or “chainsaw” EM fields.

According to some embodiment of the invention, the transmitter generates multi-frequency EM fields, for example, consisting of multiple harmonies of the base frequency f₀, for example: f₀, 2f₀, 3f₀ with potentially different strengths (see below).

According to some embodiments of the invention, the low-frequency EM tracking system comprises a receiver that analyzes the sensed profile to detect, model and compensate for the distortion.

According to an aspect of some embodiments of the present invention there is provided a shape sensor which contains magnetic shielding made of magnetic shield material to reduce the distortion of magnetic tools.

According to some embodiments of the invention, the magnetic shield material is permalloy, supermalloy, MuMetal, ferritic stainless steel (such as 17-4) or any other material of high magnetic permeability.

According to some embodiments of the invention, the magnetic shield is a thin film (for example, permalloy) located below each sensor element.

According to some embodiments of the invention, the film also serves as a mechanical stiffener for the FPC.

According to some embodiments of the invention, the magnetic shield is a film wrapped around the working channel.

According to some embodiments of the invention, the magnetic shield is a braid made of a material of high magnetic permeability such as permalloy or ferritic stainless-steel (such as 17-4).

According to some embodiments of the invention, the working tools are passed through a working channel and the working channel itself is made of high magnetic permeability material, such as permalloy, supermalloy, MuMetal, or even ferritic stainless-steel (such as 17-4).

According to some embodiments of the invention, the sensor elements are calibrated to account for the surrounding magnetic materials.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of an exemplary system for position and/or shape-tracking of an elongated device, according to some embodiments of the invention;

FIG. 2a is a schematic representation of a part of an exemplary elongated device with an exemplary curve/shape sensor comprising an array of sensor elements, according to some embodiments of the invention;

FIG. 2b is a schematic representation of a tracked portion of an exemplary elongated device with an exemplary curve/shape sensor comprising an array of sensor elements and a plurality of virtual auxiliary curve points, according to some embodiments of the invention;

FIG. 3 is a schematic representation of an exemplary representation of a distorted curve and a corrected curve, according to some embodiments of the invention;

FIG. 4 is a schematic diagram illustrating Distortion Level vs. Tracked Position with a tool inside a tracked endoscope's working channel, according to some embodiments of the invention;

FIG. 5 is a flowchart of an exemplary method for position and/or curve/shape-tracking of an elongated device, according to some embodiments of the invention;

FIG. 6 is a flowchart of an exemplary method for working channel tool calibration, according to some embodiments of the invention;

FIG. 7 is a theoretical diagram of the amount of distortion sensed along the elongated device, according to some embodiments of the invention;

FIG. 8 is a schematic representation of exemplary shielding for sensors, according to some embodiments of the invention; and

FIG. 9 is a schematic representation of another exemplary shielding for sensors, according to some embodiments of the invention.

### DESCRIPTION OF SPECIFIC EMBODIMENTS OF THE INVENTION

The present invention, in some embodiments thereof, relates to system and methods for position and/or shape-tracking of an elongated device and, more particularly, but not exclusively, to system and methods for performing corrections on position and/or shape-tracking of an elongated device, optionally taking under consideration electromagnetic distortions.

### Overview

An aspect of some embodiments of the invention relate to a system for position tracking and/or shape-tracking of an elongated device. In some embodiments, the system is configured to calculate an overall accurate and smooth position and/or curve tracking, while, when and if necessary, perform corrections during the calculations of the position and/or shape-tracking. In some embodiments, possible corrections are required due to one or more of: intrinsic system noise, for example due to irregular movements “sensed” by the system (also referred as “jitters”), and due to magnetic interferences and/or distortion that cause interferences and/or distortions in the “sensed” information received by the system. In some embodiments, the system includes a shape sensor to estimate a position and orientation of the device at a plurality of points along the device. In some embodiments, the system includes a processor configured to allocate local energy functions to each of the plurality of points. In some embodiments, the local energy functions depend on the estimated position and orientation of device at this point. In some embodiments, optionally, the local energy function incorporates relevant mechanical and sensor measurement constraints for the point. In some embodiments, a segmental energy function is at least one selected from the group consisting of: a. at least one energy function corresponding to the tracking approximation of a sensor point; b. at least one energy function corresponding to the length approximation between adjacent points; c. at least one energy function corresponding to the distortion approximation of a sensor point; d. at least one energy function corresponding to the orientation difference between adjacent points; c. at least one energy function corresponds to the twist difference between adjacent points; f. at least one energy function corresponds to the smoothness/curvature difference between adjacent points; and g, wherein at least one energy function corresponds to the motion difference between a point and a motion model of that point. It should be understood that each of the abovementioned energy functions alone is enough, and that a combination thereof is could also be used for the correction of the position and/or shape-tracking, if necessary. In some embodiments, the system then generates a resultant unified energy function for the full shape and position of the entire tracked portion of the device. In some embodiments, the unified energy function is constructed based on the allocated local energy functions and segmental energy functions that relate to constraints of mechanical properties of the device, with respect to relative locations and orientation of adjacent plurality of the predetermined points. In some embodiments, the system then calculates a fully localized curve along the tracked portion of the device by minimizing the energy function.

An aspect of some embodiments of the invention relates to a method for resolving distortions and/or reducing noise (also referred to as jitter) in a position and/or shape sensing detector array, such as an electromagnetic (EM) shape sensor which is made highly immune to electromagnetic metal distortion. In some embodiments, the method comprises the use of an array of a plurality of sensor elements, a processing unit, and algorithms by which a curve is mathematically fitted. In some embodiments, optionally, the combined measurements from all, or some, sensor elements in the array are used to model certain distortion conditions such that the final solved curve through the sensor elements is almost unaffected by distortion. Certain smoothness and length constraints are additionally imposed on the fully tracked curve such that the final solved curve is even further immune to distortion and less jittery. In the case of a position and/or shape-tracked endoscope (or other tubular device), distortion by tools introduced through the endoscope's working channel are modeled and compensated for, to both provide distortion immunity for the shape-tracked endoscope, as well as for tracking the position of the introduced tool inside the working channel. In addition, optional magnetic shielding is incorporated into such endoscope to reduce the distortion effect of tools inside the working channel. The energy-based framework disclosed herein is general and supports the simultaneous solving of position and orientation of a plurality of sensors. It allows sharing of distortion information between all solved sensors and combines additional constraints, which provides increased accuracy.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary System

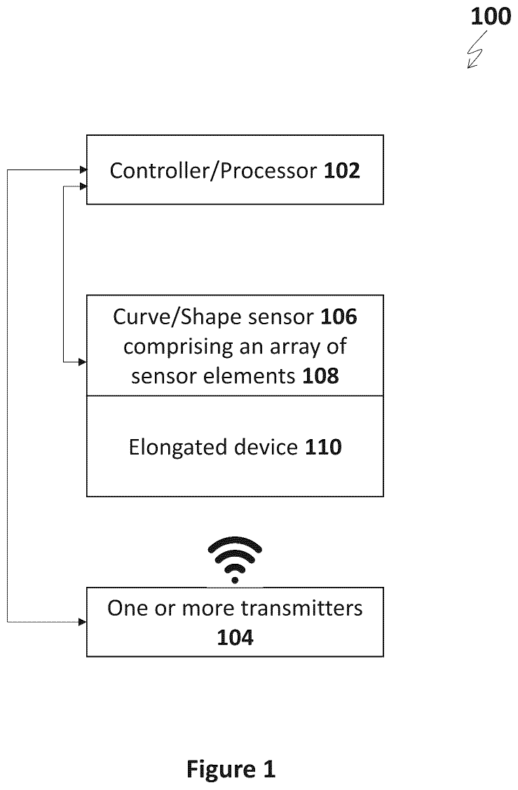

Referring now to FIG. 1, showing a schematic representation of an exemplary system for position and/or shape-tracking of an elongated device, according to some embodiments of the invention.

In some embodiments, exemplary elongated devices can be one or more of an endoscope, a catheter and any other interventional device. In some embodiments, exemplary elongated devices can be elongated non-medical devices that require to be tracked.

In some embodiments, an exemplary system 100 comprises a controller 102 comprising a processor, one or more transmitters 104 and a curve/shape sensor 106 comprising an array of sensor elements 108. In some embodiments, the curve/shape sensor 106 is mounted on an elongated device 110. In some embodiments, an elongated device 110 already comprises the curve/shape sensor 106 built therein.

In some embodiments, the one or more transmitters 104 are configured for transmitting one or more electromagnetic fields.

In some embodiments, the elongated device 110 is configured to be manipulated to various positions and/or shapes, which are sensed by the curve/shape sensor 106. In some embodiments, optionally, the elongated device 110 comprises a working channel (not shown) configured to receive one or more instruments and/or tools.

In some embodiments, the curve/shape sensor 106 is configured to detect magnetic fields in multiple locations along the elongated device 110, according to the number and location of the sensor elements 108 in the curve/shape sensor 106; for example, the one or more magnetic field generated by the one or more transmitters 104.

In some embodiments, the controller 102 comprises instructions to algorithmically calculate a curve and/or position of the elongated device 110 based on the sensed electromagnetic fields values by the array of sensor elements 108 in the curve/shape sensor 106, for example, relative to the one or more transmitters 104 and/or to a predetermined frame of reference (not shown).

For example, during operation of the elongated device 110, the elongated device 110 is inserted into a body lumen, while the one or more transmitters 104 generate magnetic fields. The array of sensor elements 108 in the curve/shape sensor 106 detect the local magnetic fields along the elongated device 110. The processor in the controller 102 calculates a shape of the elongated device 110, and, in some embodiments, a position of the elongated device 110, for example, relative to the one or more transmitters 104 and/or to a predetermined frame of reference.

In some embodiments, optionally, the system 100 comprises one or more transmitters (not shown) integrated along the elongated device 110 instead of the one or more transmitters 104.

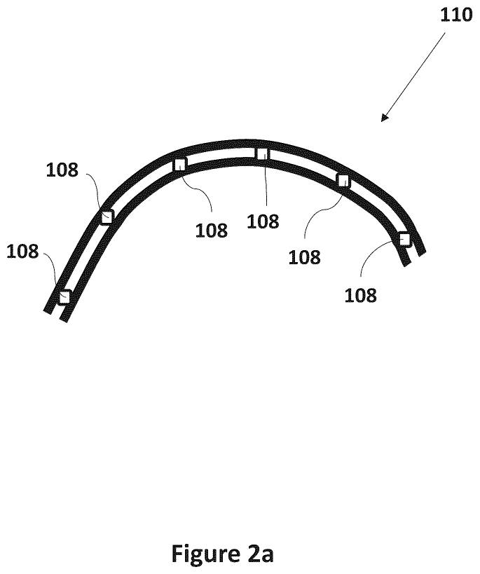

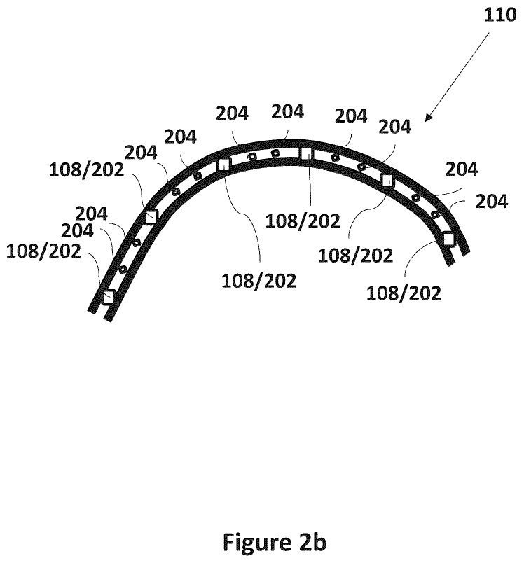

Referring now to FIG. 2a, showing a schematic representation of a part of an exemplary elongated device with an exemplary curve/shape sensor comprising an array of sensor elements, according to some embodiments of the invention. Same reference numbers are used for same parts in all Figures. In some embodiments, as mentioned above, a curve/shape sensor 106 comprising an array of sensor elements 108 is either mounted on an exemplary elongated device 110 or already incorporated into an exemplary elongated device 110.

In some embodiments, each of the sensor elements 108 in the curve/shape sensor 106 is configured to sense transmitted magnetic fields (either from the one or more transmitters 104 or from the integrated transmitters). For example, the one or more transmitters 104 transmit multiple magnetic fields with known frequencies and intensities. In some embodiments, the shape of the elongated device 110 is estimated by finding the position of each sensor element 108, according to the sensed magnetic fields. In some embodiments, additionally, a curve is “fitted” (see below for more information regarding the “fitting” of a curve) through two or more locations of the sensor elements 108 along the elongated device 110. In some embodiments, a potential advantage of the methods, as will be described below, is that it potentially overcomes errors and inaccuracies of the actions disclosed in this paragraph regarding the calculation of the shape of the elongated device 110.

For example, the one or more transmitters 104 include N_{c} coils to generate corresponding N_{c} different magnetic fields, and the elongated device 110 includes Nₓ sensor elements 108. In some embodiments, the one or more transmitters 104 may include other means to generate different magnetic fields, such as, for example, various formations of rotating magnets. In some embodiments, without being bound to theory, the theoretical resulting magnetic fields Xi that should be sensed at the location and orientation of sensor i are:

$${X}_{i} = {R}_{i}^{T} B ( {\vec{r}}_{ι} ) ,$$

where Rᵢ is the three-dimensional orientation of the sensor element i with respect to the one or more transmitters 104, and B(\vec{rᵢ}) are the generated magnetic fields at the three-dimensional location \vec{rᵢ} of the sensor element i with respect to the one or more transmitters 104.

In the case of 5-DOF sensors, the measured fields are

$${X}_{i} = {z}_{i}^{T} B ( {\vec{r}}_{i} ) ,$$

Where zᵢ is the normalized (column vector) z axis of the i-th sensor.

In some embodiments, for each of the Nₓ sensor elements, estimated six-dimensional values of location and orientation are found by minimizing an energy function Eᵢ, that depends on the difference between the resulting magnetic fields Xᵢ and the measured magnetic fields Xᵢ^{measured} by the sensor element i. For example, Ei has the form:

$${E}_{i} = \frac{1}{2} {\vert {R}_{i}^{T} B ( {\vec{r}}_{ι} ) - {X}_{i}^{measured} \vert}^{2} .$$

Note that Xᵢ^{measured} may be corrected to account for sensor calibration, that is, it may be a calibrated version of the raw fields measured by the sensor.

Also note that the sensor may not measure the generated fields Xᵢ directly. Instead, it may measure 3D magnetic field vectors over time, which after using Discrete Fourier Transform (DFT) or similar spectral decomposition methods are transformed into a signed intensities matrix Xᵢ which reflects the intensities of the multi-frequency generated fields at the sensor position.

Throughout the disclosure, sensor measurements/sensor readings may refer to the computed matrices Xᵢ of the transmitted fields at the sensor position and orientation rather than to the raw measured magnetic fields.

In some embodiments, after the six-dimensional locations and orientations are found for the sensor elements 108, a curve is fitted to pass through all the sensors. However, such a curve may be insufficiently accurate. In some embodiments, the reasons why such curve might be insufficiently accurate are one or more of: the found positions and orientations of the sensors may be erroneous, because of noisy or faulty measurements by the sensor elements 108 and/or because of magnetic field distortions; and second, without further constraints, the curve shapes between the sensor elements 108, which may be interpolated arbitrarily and/or based on some predetermined assumptions, may be incorrect. Additionally or alternatively, the calibration of the sensor elements 108 may be slightly erroneous which can introduce inaccuracy to the sensor's solved position and orientation, and thus to the solved curve/shape.

Therefore, the fully-calculated curve of the elongated device 110 may have illogical properties. For example, the total length of the curve may not match the device length known from manufacturing or from calibration of the elongated device 110. Such constraints that relate to the entire shape are not considered in the calculation, since the values for each sensor are solved separately.

In some embodiments, in order to solve such issues, a method for solving the entire curve and/or location of the elongated device 110, while considering various constraints that reduce the resulting shape inaccuracies is performed.

Referring now to FIG. 2b, showing a schematic representation of a tracked portion of an exemplary elongated device with an exemplary curve/shape sensor comprising an array of sensor elements and a plurality of virtual auxiliary curve points, according to some embodiments of the invention. In some embodiments, an exemplary elongated device 110 representation may represent dynamic position and/or a curve of the tracked portion of the elongated device 110. In FIG. 2b, an elongated device 110 representation includes sensor curve points 202, representing locations of the sensor elements 108 along the elongated device 110, and/or located in potentially known locations and/or intervals along the tracked portion of the elongated device 110, where magnetic field values are measured. In some embodiments, optionally, between one or more couples of adjacent sensor curve points 202, there may be a pre-determined amount of virtual auxiliary curve points 204, at predetermined locations and/or intervals along the representation of the elongated device 110. In some embodiments, sensor curve points 202 and virtual auxiliary curve points 204 are used for calculation of a curve and/or position of the tracked portion of the elongated device 110.

Exemplary Principles Applied in an Exemplary Position and/or Shape-Tracking of an Elongated Device

As mentioned in the “FIELD AND BACKGROUND OF THE INVENTION” section above, static EM distortion effects can be modeled in a mapping or calibration process. However, dynamic EM distortion which accounts for distortion fields created by dynamically moving distorting objects requires a different solution and are difficult to be addressed in a pre-calibration or mapping process.

In a clinical environment, dynamic EM distortion can be caused for example by a C-arm which is centered at the patient and is rotated and moved during procedure. Inside a tracked endoscope (or other tubular device), tools made of magnetic metals are introduced into the endoscope's working channel and may create dynamic EM distortion while they are moved and manipulated inside the endoscope's working channel. Such tools may be for example: biopsy tools (such as forceps, needles, cytology brushes); endoluminal ultrasound devices (such as REBUS, IVUS); other endoluminal imaging (such as OCT and spectroscopy devices); ablation devices (such as RF probes, Microwave probes, cryoablation devices, drug delivery needles and probes, brachytherapy devices and seeds, laser and light fiber optics); stents and stent placement tools; clot and foreign-object retrieval tools (such as mechanical baskets, electronic devices, suction microcatheters); embolization devices (such as coils, catheters, and aneurism management devices); fiducials and their placement mechanisms; flexible endoluminal surgical tools; lithotripsy and other types of therapeutic ultrasound devices. Other forms of dynamic distortion may be caused by electric cauterization. Yet other forms of dynamic EM distortion may be caused by positioning an EM sensor array in close proximity to metallic or electronic implanted devices, such as pacemakers or electrostimulation devices, orthopedic implants, stents and prosthetics, or dental implants. This dynamic distortion might impact the accuracy of the solved position and orientation of an EM sensor inside the endoscope. In an endoscopic procedure, this may impact the tracking accuracy of the endoscope during a biopsy or therapeutic process which may impact the yield. It is therefore desired for an EM tracking system to be partially immune, optionally highly immune, optionally completely immune to EM distortion, especially in a clinical use-case.

In some embodiments, the exemplary system 100 performs the following exemplary method for resolving distortions in a curve/shape sensing detector array, such as an electromagnetic (EM) curve/shape sensor 106, which is potentially made highly immune to electromagnetic metal distortion. In some embodiments, the method relies on the use of the array of a plurality of the sensor elements 108, the processing unit 102, and algorithms by which a curve is mathematically fitted.

For example, it is possible to use as few as two sensors to solve a curve between them with the methods described herein. For example, known mechanical distances, smoothness, minimal-twist, etc., between those two sensors are all beneficial constraints which can provide a better curve solution between the two sensors. In some embodiments, the position of the tip of the elongated device can be extrapolated after the most distal sensor (for example, 3 mm after the location of the most distal sensor on the elongated device) using virtual points (as described herein elsewhere) which are subject to the same energy constraints. In some embodiments, a potential advantage of extrapolating the tip using virtual points, which are subject to the same energy constraints, is that it potentially provides a much better extrapolation compared to “naïve” extrapolation (which just extrapolates a spline without any additional mechanical constraints).

In some embodiments, the combined measurements from all sensor elements 108 in the array are used to model certain distortion conditions such that the final solved curve through the sensor elements 108 is almost unaffected, highly unaffected, or less affected by distortion. In some embodiments, certain smoothness and length constraints are additionally imposed on the fully tracked curve such that the final solved curve is even further immune to distortion. In some embodiments, a potential advantage of the method is that it potentially provides a final solved curve that is smoother and experiences less jitter, which allows to generate weaker electromagnetic fields in the EM transmitter, as well as decreasing the filters and latency in the EM curve tracking solution. In some embodiments, alternatively, for identical fields, the effective tracking volume can be extended.

In some embodiments, in the case of a curve/shape-tracked manual or robotic endoscope (or other tubular device), distortion by tools introduced through the endoscope's working channel are modeled and compensated for, to both provide distortion immunity for the curve/shape-tracked endoscope, as well as for tracking the position of the introduced tool inside the working channel. In some embodiments, in addition, optional magnetic shielding is incorporated into such an endoscope to reduce the distortion effect of tools inside the working channel.

In some embodiments, in the case of a position and/or curve/shape-tracked probe, inserted into a working channel of an endoscope to provide position and/or curve/shape tracking for that endoscope, distortion by surrounding metals in the endoscope (such as the endoscope's braid, links, pull-wires or other metallic objects) are modeled and compensated for, to both provide distortion immunity for the curve/shape-tracked probe, as well as for potentially tracking the position of the introduced probe inside the endoscope's working channel. In some embodiments, a potential advantage of the method is that the energy-based framework disclosed herein is general and supports the simultaneous solving of position and orientation of a plurality of sensors, which allows sharing of distortion information between all solved sensors and allows combining additional constraints, which provides increased accuracy and reduces noise (jitter).

In general, compensating for dynamic EM distortion is challenging since the characteristics of the distorter, such as size and material, position and the orientation of the distorter relative to the EM transmitter or to the receiver, may be unknown. Furthermore, the very presence of an EM distorter may not be known.

In some embodiments, as a first step, the EM distortion is detected by the system. In some embodiments, with EM curve/shape sensing, the distortion can be detected along the fully tracked curve/shape such that it can be indicated where along that curve a distortion is present. In some embodiments, this information is used, for example, to ignore or just partially ignore (by decreasing its weight) a certain distorted sensor element 108 along the tracked curve and interpolate its position and orientation using its neighboring sensors. In some embodiments, alternatively, it can be used to model the distorter and solve all sensors' positions and orientations (including the distorted sensors) according to the modeled distortion fields.

In general, with a single tracked EM sensor, detecting and compensating for EM distortion is even more challenging, since the number of measurements is rather small (for example, with a 3-fields transmitter and a 3-coils sensor the number of measurements is 9, which is usually used to solve for 6-DOF). However, in some embodiments, in the setting of EM curve/shape sensing, the number of measurements is increased in proportion with the number of sensor elements used along the tracked length. In some embodiments, additionally, certain mechanical constraints can be imposed on the solved position and/or curve/shape of the tracked device. For example, the distance between neighboring EM sensors along the sensed length can be known or calibrated in advance. As another example, in a clinical setting, it may be known that a curve/shape tracked device is mechanically stiff up to a certain extent, that is, it cannot mechanically bend below a certain bend radius and/or cannot twist, either at all or above a certain twisting angle. In some embodiments, these constraints (length, smoothness, etc.) are used to improve the accuracy of the fully tracked curve, even under EM distortion, but also without the presence of EM distortion. In some embodiments, these constraints are used to detect, model and compensate for EM distortion as well as to improve the accuracy and reduce the noise of the fully tracked curve in general, for example, by always solving for a mechanically plausible curve (for example, smooth curve) of correct lengths between sensors (as recorded in calibration).

In some embodiments, as previously mentioned, an EM curve/shape sensing is based on a plurality of points along the tracked device, for which corresponding local measurements of magnetic fields can be performed, and/or for which corresponding mechanical constraints can be imposed; and/or based on virtual point(s) positioned between known positions of sensors, as disclosed herein elsewhere. In some embodiments, the use of EM curve/shape sensing can therefore assist in the detection, modeling, and compensation of EM distortion.

In some embodiments, as mentioned above, an exemplary EM curve sensing device consists, for example, of a plurality of tracked EM sensors 108 on a single elongated device 110, or, for example, a single elongated EM curve sensor, for example as described in PCT application publications WO2023/089623 or WO2023089624, or in U.S. Pat. No. 11,712,309B2.

In some embodiments, also as mentioned above, the exemplary EM curve/shape sensor 106 consists of a sensor-array made of multiple EM sensor elements 108, each of which are individually tracked. In some embodiments, a curve is mathematically fitted between the plurality of sensor elements 108 based on their magnetic field measurements to provide position and/or full curve tracking of the EM curve/shape sensor. In some embodiments, a curve is calculated for the entire length of a tracked portion of the device, which minimizes the measurement errors along tracked portion of the device, for example together with errors relating to other mechanical constraints, thus providing a more stable and accurate curve tracking.

In some embodiments, position and/or curve/shape tracking is achieved by an array of discrete measurement points, such as grading in a fiber optic detector, for which a source of distortion exists, such as light, heat, or distance, or curvature, or corruption of a grading, or of the data transferred.

In some embodiments, position and/or curve/shape tracking is achieved by detecting a series of points in an image of an object, such as a fluoroscopic image or a camera image, for which a source of distortion and/or noise exists, such as obscured areas of the image, or distortion of field of view, or distortion of exposure.

In some embodiments, other discrete-point detection methods are utilized to detect a curve, and other forms of distortion may prevent some portion of these points from being correctly measured (with respect to a theoretical model of the measurements). Throughout the explanations herein, focus is given to EM curve/shape sensing and to corresponding distortion modeling and compensation methods. However, most of the methods described herein are applicable to other kinds of curve/shape sensing solutions, such as fiber optics shape sensing, visual shape sensing etc., and may provide a better solution for these methods, in terms of increased accuracy and reduced noise.

In some embodiments, solving the full curve of the EM curve/shape sensor is achieved by first solving a 6-DOF position and orientation of each of the individual sensor elements. For example, in the case of 3-fields EM transmitter and 3D sensor elements (for example, each being a 3D digital magnetometer), by sampling a short window of samples over time (for example, sampling at 1 Khz a window of 30 samples which amounts to 30 milliseconds) the three transmitted fields can be decomposed, for example, using Discrete Fourier Transform (DFT), correlation methods, linear solver methods or any other suitable methods. In some embodiments, this provides 3D measurements of the three individually transmitted fields for each sampling window, which amounts to nine measured values for each sensor element. In some embodiments, these values are used to solve for 6-DOF of each of the sensor elements inside the EM curve/shape sensor. In some embodiments, using the 6-DOF (or 5-DOF) position and orientation of each of the sensor elements, a curve is fitted between the tracked elements which respects the tracked positions and orientations of each of the tracked discrete sensor elements. For example, a cubic curve can be fitted between each two neighboring sensor elements (that is, along each tracking segment) such that it starts at the first sensor element's position and direction and finishes at the second sensor element's position and direction. The rest of the orientation (that is, the roll of the sensor) can be interpolated externally, for example using linear interpolation or quaternion interpolation (for example spherical linear interpolation (Slerp)). In some embodiments, a potential advantage of ensuring smooth interpolated transitions between each sensor to its neighboring sensor is that it potentially guarantees the fully tracked curve to be smooth everywhere along its length.

In some embodiments, one potential problem which may arise from using the individually solved positions and orientations of each of the sensor elements is that any error in position and orientation of any of the individually tracked sensor elements may be directly reflected in the final computed curve, since each of the elements serves as a key-point inside that curve. Therefore, in some embodiments, instead of directly using the position and orientation of each sensor element to interpolate the final curve, a full curve is searched which fits the tracked positions and orientations of the individual sensor elements under certain smoothness and length constraints. In some embodiments, fitting the curve is done in an optimization process, for example by minimizing the following energy function:

$$E ( \{ {r}_{i} , {q}_{i} \} ) = {E}_{track} ( \{ {r}_{i} , {q}_{i} \} ) + {E}_{smooth} ( \{ {r}_{i} , {q}_{i} \} ) + {E}_{length} ( \{ {r}_{i} , {q}_{i} \} ) ,$$

where {rᵢ}, {qᵢ} are the corrected positions and orientations of the discrete sensor elements inside the EM curve/shape sensor. In some embodiments, a curve is then fitted as described above between the corrected positions and orientations of the sensing elements rather than the initially solved ones. In some embodiments, E_{track} measures the difference between the corrected searched positions and orientations and the measured (individually solved) positions and orientations: { [symbol] }, { [symbol] }. Eₛₘₒₒₜₕ computes the smoothness error of the solved rᵢ, qᵢ—that is, how smooth is the final curve. It can do so for example by constructing the full final interpolated curve and summing the absolute values of its second derivatives. In some embodiments, E_{length} computes the length error of the solved rᵢ, qᵢ—that is, how accurate are the lengths between neighboring sensor elements, or between virtual points, in the final curve. In some embodiments, it can do so for example by constructing the full final interpolated curve and summing the differential length elements between neighboring sensors, comparing them with known or pre-calibrated distances between these sensors. In some embodiments, E_{track} ensures that the corrected positions and orientations match the individually solved ones. However, when a certain sensor element is known to be distorted (for example, if a high amount of distortion field is detected for a certain sensor element), then, in some embodiments, E_{track} uses weights to decrease the error of that specific sensor, such that the requirement that the corrected position and orientation rᵢ, qᵢ would match the individually computed ones \hat{r}ₗ, \hat{q}ₗ would be relaxed. In some embodiments, the corrected position and orientation would then be naturally computed using smoothness and length constraints based on neighboring sensors inside the optimization process. In some embodiments, a potential advantage of doing this is that this potentially guarantees a final curve which complies to known smoothness and length constraints regardless of the present of distorters. In some embodiments, additionally, this potentially leads to an increased accuracy even in the presence of distorters and even without modeling them.

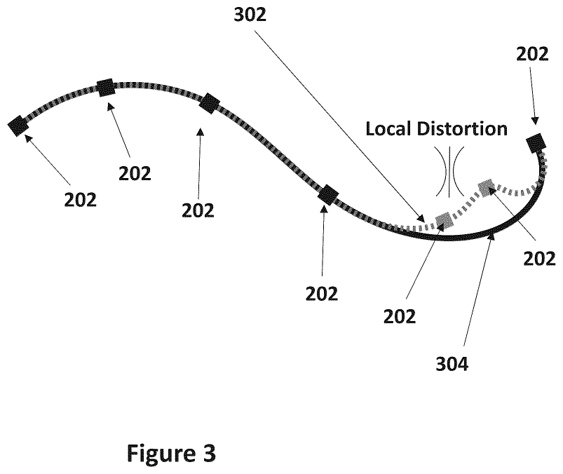

In some embodiments, in a clinical setting, when introducing a working channel tool into an EM curve/shape tracked endoscope, it may be the case that the tip and/or body of the tool is magnetic and so it may create local EM distortion in its close proximity (for example, in a radius of from about 3 mm to about 5 mm or from about 5 mm to about 10 mm or from about 10 mm to about 20 mm around it). In this case, when introducing the tool, one or more of the sensor elements 108 along the EM curve/shape sensor may be highly or partially affected by the EM distortion caused by the tools' tip and/or body, thereby potentially causing their individually solved position and orientation [symbol] , [symbol] to be inaccurate. In this case, as described above, the presence of distortion will be detected for these two sensors. In some embodiments, due to their high amount of distortion, the method performed by the system will decrease their corresponding weight in the E_{track} energy, causing the curve fitting optimization process to solve for their corrected position and orientation rᵢ, qᵢ more based on Eₛₘₒₒₜₕ, E_{length} so that they will be much more accurate and so that the final solved curve would be much less affected by their initial inaccuracy due to the local EM distortion, as illustrated for example in FIG. 3.

Referring now to FIG. 3, showing a schematic representation of an exemplary representation of a distorted curve and a corrected curve, according to some embodiments of the invention. FIG. 3 shows a dashed gray distorted curve 302 and a continuous black corrected curve 304 along a plurality of sensed sensor curve points 202 (not to confuse those points with the actual location of the sensor elements 108 along the elongated device 110). In some embodiments, distorted curve 320 is an initially solved curve based on initial positions and orientations with local distortion at the distal part of the curve/shape sensor. In FIG. 3 is shown that two distal sensor elements are distorted due to local distortion (for example, tool introduced into working channel). In some embodiments, a corrected curve 304 is a curve calculated with smoothness and length constraints, mostly ignoring the distorted sensors and/or compensating for the distortion fields.

In some embodiments, the detection of EM distortion is done by estimating the relative 6-DOF or 5-DOF tracking approximation error which remains after finding the, optionally, best (optimal) position and orientation which approximates the magnetic field measurements. In some embodiments, each sensor element's position and orientation is solved in an optimization process by finding the, optionally, best (optimal) position and orientation such that the expected (calibrated theoretical) fields approximate the actually measured magnetic fields. In some embodiments, as described above, with a 3-fields transmitter, each 3D sensor element 108 (optionally a magnetometer sensor) measured nine values from which a 6-DOF solution needs to be found. In some embodiments, since nine values exceed the number of searched parameters (six or five parameters) then the problem is overdetermined and in many cases of distortion field, the solution will not perfectly fit. That is, a position and orientation found will have a small fitting error (for example, larger than 1%, or larger than 3%), such that the theoretically modeled magnetic field at that position and orientation fit the measurements to that fitting error. This means that no position and orientation can be found which matches the measured fields to a small error, for example smaller than 1%, or smaller than 3%.

In some embodiments, the remaining relative error (that is, the difference between the expected fields and the measured fields at the optimal found position and orientation) can be used as an approximation for the level of EM distortion at the specific sensor.

In some embodiments, as described herein, the approximated distortion level can be used to compute weights in the energy function E_{track} to indicate the curve to interpolate a distorted sensor's position and orientation using neighboring sensors rather than using its solved inaccurate position and orientation. For example, a sigmoid function can be used to produce a weight which decreases from 1 to 0 as the relative EM fitting error grows (the weight reflects the level of certainty in the EM solution, to it is inversely correlated to the EM fitting error). For example, the level of certainty can be 1 for a 0% fitting error, can be 0 for a 100% fitting error, and can be 50% for a 3% fitting error.

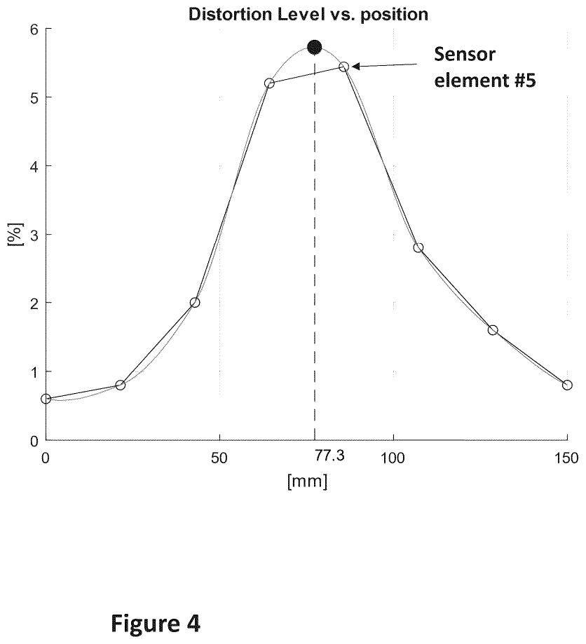

In some embodiments, in a clinical setting, the detection of distortion of one or more specific sensor elements along the EM shape sensor is used to track the progression of clinical tools inside an EM curve/shape tracked endoscopic working channel. In some embodiments, the final solved curve will then mostly ignore the initial solutions of the distorted sensor elements such that their corrected positions and orientations according to the final fitted curve will be much more accurate. In some embodiments, the detected distortion level along these sensors can be used to track the position of a tool inside the endoscope's working channel. In some embodiments, this position can be visualized and displayed to the user for example as a 3D tool rendered inside the virtual working channel on a monitor. In some embodiments, this information can serve a physician while they insert or pull a tool inside an endoscope's working channel. In some embodiments, the tool's position inside the working channel can be tracked for example by finding the smooth maximum point along the curve of distortion level vs. tracked length, as shown for example in FIG. 4.

Referring now to FIG. 4, showing a schematic diagram illustrating Distortion Level vs. Tracked Position with a tool inside a tracked endoscope's working channel, according to some embodiments of the invention. For example, eight sensor elements are used, each experiences a different level of distortion due to the introduced tool. The tool's tip position is tracked using smooth maximum location (for example, at 77.3 mm along the endoscope's tracked curve).

In FIG. 4, the distortion levels of eight individual sensor elements are shown. Each of the sensor elements is known to be located at a certain position (length) along the tracked curve of an endoscope. In some embodiments, when a tool is introduced into the endoscope's working channel, it may create EM distortion fields which are sensed by some of the sensors. As shown in FIG. 4, the sensor which is most affected by the introduced tool is, for example, sensor element #5 which experiences a distortion level of 5.44%. A smooth spline interpolation can, for example, be used between the distortion levels of the different sensor elements to then find a smooth maximal distortion level, in between the sensor elements. In some embodiments, the maximum point is found at position 77.3 mm and its predicted value is 5.73%. In some embodiments, the tool's tip is then assumed to be located at position 77.3 mm along the tracked curve of the endoscope.

To further generalize the solution described above, in some embodiments, instead of first solving the initial positions and orientations of the sensor elements (with the distortion fields) prior to fitting the curve, a curve can be fitted directly onto the magnetic field measurements with the imposed constraints. In this framework the conversion from magnetic field measurements to 6-DOF or 5-DOF positions and orientations of the sensor elements is done as part of the curve fitting optimization process, in combination with the imposed constraints. In this case the initial (and potentially distorted) [symbol] , [symbol] are no longer computed in advance, and a curve is fitted directly to minimize an energy function similar as E(rᵢ, qᵢ) defined above, only now E_{track} (rᵢ, qᵢ) expresses the approximation error between the solved rᵢ, qᵢ and the measured magnetic fields, instead of comparing to the initial [symbol] , [symbol] .

While quaternions and Euler angles are usually equivalent, in the case of 5-DOF, it is usually simpler to use Euler angles as the rotational degrees of freedom, θᵢ=(αᵢ, βᵢ, γᵢ). When the “roll” degree of freedom (rotation around the \hat{z} axis) is absent, one of the angles can simply be dropped. For example, using the ZYX convention, the α angle holds the roll degree of freedom. Then each sensor can be fully described by x, y, z, β, γ.

In some embodiments, E_{track} can then be defined as a 6-DOF or 5-DOF solver energy function, only now it is combined inside a full curve fitting framework which stabilizes it even in cases of noise and/or distortion:

$${E}_{track} ( \{ {r}_{i} , {q}_{i} \} ) = \frac{1}{2} {\sum }_{i = 1}^{N} {W}_{i} \cdot {‖ {R ( {q}_{i} )}^{T} \cdot B ( {r}_{i} ) - {X}_{i} ‖}^{2} .$$

Here N is the number of sensor elements inside the EM shape sensor, Wᵢ is the tracking weight of each sensor element—that is, how reliable is the magnetic measurement of that sensor. In some embodiments, Wᵢ can for example be precomputed in a preliminary step in which each individual sensor element's position and orientation is computed individually to estimate the distortion level of each sensor element. In some embodiments, Xᵢ is the (calibrated) magnetic field measurements for sensor element i. In case of an N_{c}-fields transmitter and 3D sensor elements, the magnetic field measurement Xᵢ can be represented as a 3×N_{c} matrix (containing N_{c} 3D magnetic field measurements). B(r) is the calibrated theoretical (predicted) magnetic fields in transmitter coordinate system which are expected at position rᵢ and may be represented as a 3×N_{c} matrix, R(q) is a matrix which projects the theoretical fields at rᵢ from transmitter to sensor coordinates based on sensor orientation qᵢ, and it may be a 3×3 rotation matrix in the case of a 6-DOF solver or a 3×1 vector in the case of 5-DOF solver. For a single tracked sensor element,

$${E}_{track}^{i} ( {r}_{i} , {q}_{i} )$$

represents a 6-DOF or 5-DOF solver energy function, but in the context of multiple sensors mixed together into a single energy function E_{track} and tied together through imposed constraints (Eₛₘₒₒₜₕ, E_{length}) the final solved positions and orientations rᵢ, qᵢ are much improved even under distortion.

In some embodiments, Wᵢ can for example be computed in an iterative process. For example, the curve can be solved with Wᵢ=1. After solving the entire curve (using the methods described herein), tracking error can be estimated at each of the solved sensors along the curve. This tracking error can then be used to re-compute Wᵢ (for example, using a sigmoid function as described above). With the newly computed Wᵢ, the curve can be refitted. This process can be repeated several iterations (for example, 3-4 iterations) where in each iteration Wᵢ are updated. This process is similar to RANSAC (Random sample consensus) where weights are updated iteratively to account for outliers (in this case, highly distorted sensors) in the data.

In some embodiments, methods for full position and/or curve/shape-tracking of an elongated device, for example a catheter and/or another device for endoscopic interventional procedures are performed by the system 100. In some embodiments, methods use virtual auxiliary curve points between sensor elements of the device, and/or include solving of the entire shape of the device with a unified energy function, based on mechanical constraints. In some embodiments, such methods may constitute a solution to insufficiently accurate curve estimations. In some embodiments, inaccurate curve estimations may be resulted from noisy or faulty measurements by the sensors, from magnetic field distortions and/or from large distances between the sensors on the device, that prevent sufficiently accurate curve interpolations.

Since in some previous methods, the curve estimation is based on separate calculations of position and orientation for each sensor element, the previous methods could not incorporate constraints that relate to the entire curve/shape of the device. In some embodiments, a potential advantage of the methods disclosed herein is that the methods potentially solve such issues, by solving the entire curve and/or location of the device, while considering various constraints that may reduce the resulting curve/shape noise and/or inaccuracies.

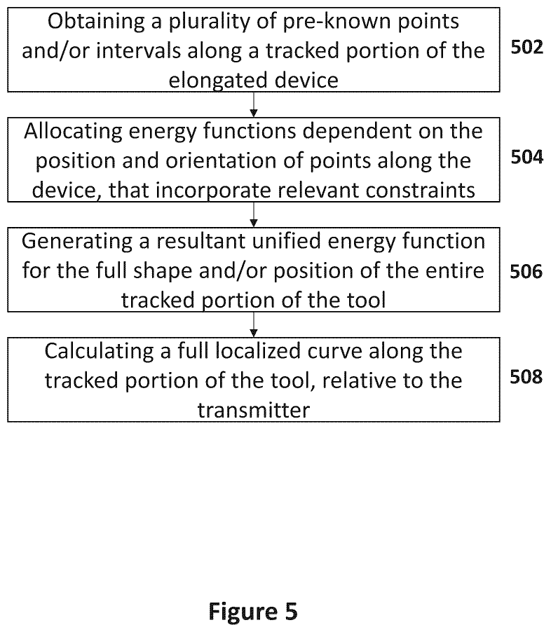

Referring now to FIG. 5, showing a flowchart of an exemplary method for position and/or curve/shape-tracking of an elongated device 110, according to some embodiments of the invention. In some embodiments, a shape and/or position of the elongated device 110 is tracked dynamically by fitting a shape that is the most energetically efficient, based on various constraints. In some embodiments, as indicated in block 502, processor/controller 102 obtains a plurality of pre-known points and/or intervals along a tracked portion of the elongated device 110, such as sensor points 202 and curve points 204 in known intervals. In some embodiments, as stated above, between every two adjacent sensor elements, represented by points 202, there may be a pre-determined amount of virtual curve points 204, at predetermined locations and/or intervals along the tracked portion of the elongated device 110.

In some embodiments, as indicated in block 504, controller/processor 102 allocates for each sensor point 202 a local energy function dependent on the position and orientation of the elongated device 110 at this point, that incorporates relevant constraints for the point or for the type of point. For example, the energy function for each sensor point 202 incorporates constraints related to the sensed magnetic field at this point, similarly to Eᵢ discussed herein. In some embodiments, controller/processor 102 optionally further allocates a weight for each local energy function, for example based on a certainty value, for example related to a certainty that a measurement is accurate. For example, a measurement value taken by a sensor element 108 may have a certain variation along a short time frame in which samples for the measurement are collected, according to which a certainty value may be determined.

For example, in some embodiments, a few different known magnetic fields with respective different frequencies are generated, for example by the one or more transmitters 104 and/or by another suitable method, for example by internal transmitters on curve/shape sensor 106. At a plurality of points along curve/shape sensor 106, the resulting magnetic field values are measured at each of the points. In some embodiments, at each point, the measurement is performed by collecting a plurality of sample measurements, for example, a few tens of sample measurements, during a sampling time of corresponding few tens of milliseconds, and obtaining a DFT or correlation computations or linear solver methods that decompose the sequence of samples taken within the sampling time into components of the different transmission frequencies. In some embodiments, in case the sample measurements vary substantially within the sampling time, the frequency decomposition methods will have a corresponding large error. In some embodiments, this error represents the certainty and/or the weight that is allocated to a certain local energy function.

In some embodiments, as indicated in block 506, controller/processor 102 generates a resultant unified energy function for the full curve and/or position of the entire tracked portion of the elongated device 110. In some embodiments, the unified energy function is constructed based on the allocated local energy functions of sensor point 202 and segmental energy functions that relate to constraints of mechanical properties of the elongated device 110, with respect to relative positions and orientation of curve points 204 and sensor points 202. For example, each point 202 or 204 have constraints related to its position and orientation relative to other points 204 and/or 202, which are incorporated into energy functions. For example, an energy function E for all N points 76 and 78, has the form:

$$E = {\sum }_{i = 1}^{{N}_{x}} {E}_{i} ( {\vec{x}}_{i} ) + {\sum }_{j = 1}^{N} {\sum }_{{k = 1}_{k \neq j}}^{N} {E}_{jk} ( {\vec{x}}_{j} , {\vec{x}}_{k} ) + {\sum }_{j = 1}^{N} {\sum }_{{k = 1}_{k \neq j}}^{N} {\sum }_{{l = 1}_{l \neq j , k}}^{N} {E}_{jkl} ( {\vec{x}}_{j} , {\vec{x}}_{k} , {\vec{x}}_{l} ) + … ,$$

where Eᵢ is discussed herein. Eⱼₖ relates, for example, to mechanical constraints between two points. Eⱼₖₗ relates, for example, to mechanical constraints between three points, etc. \vec{x}ᵢ is the state vector of sensor i, for example, \vec{x}ᵢ=(xᵢ, yᵢ, zᵢ, αᵢ, βᵢ, γᵢ) in the case of 6-DOF sensors.

For example, in some embodiments, the distance between curve points 204 and/or 202 is relatively small, for example up to about 2 millimeters, so that the sub-segments between the curve points can be regarded as linear. As mentioned herein, the large distances between sensor points 202 may prevent sufficiently accurate curve interpolations, and the inclusion of curve points 204 between points 202 potentially enable calculation of such accurate curve interpolations. In some embodiments, the linear approximation allows construction of a segmental length energy function

$${E}_{jk}^{length} ,$$

which forces the curve points to have known distances between them:

$${E}_{jk}^{length} ( {\vec{r}}_{j} , {\vec{r}}_{k} ) = \frac{{W}_{jk}^{length}}{2} {( \vert ( {\vec{r}}_{j} - {\vec{r}}_{k} \vert - {L}_{jk} )}^{2} ,$$

where j=k±1 (adjacent curve points),

$${W}_{jk}^{length}$$

are weights which may be different for each pair of curve points, and Lⱼₖ is the known length of the jk subsegment. This formula uses the smallest distance between them. It is assumed that it is a fine approximation, since the curve is rather straight along such small distances.

In some embodiments, another type of segmental/regional energy function that is constructed using the curve points, is a smoothness energy function in some

$${E}_{jk}^{smooth} .$$

In some embodiments, the smoothness energy function is constructed based on constraining the curve of the elongated device 110 to be of a physically plausible shape, which does not contain, for example, folds, creases, etc. In some embodiments, this can be obtained by minimizing the second derivative along the curve. For intuition, a curve of minimal second derivative is a straight line. The second derivative of a one-dimensional function can f(x) be computed as

$${f}^{″} ( x ) = {lim}_{Δ x \to 0} \frac{f ( x - Δ x ) - 2 f ( x ) + f ( x + Δ x )}{Δ {x}^{2}} .$$

Since the curve is known to controller/processor 102 at discretized locations, it cannot take Δx→0. Instead, it uses a linear approximation, which is sufficiently accurate, for the reasons mentioned and explained above, for example, because the curve points are sufficiently close. The energy function, generalized to three dimensions, can be defined as

$${E}_{jk}^{smooth} ( {\vec{r}}_{j} , {\vec{r}}_{k} , {\vec{r}}_{l} ) ∝ {{W}_{jk}^{smooth , x} ( {x}_{j} - 2 {x}_{k} + {x}_{l} )}^{2} + {{W}_{jk}^{smooth , y} ( {y}_{j} - 2 {y}_{k} + {y}_{l} )}^{2} + {{W}_{jk}^{smooth , z} ( {z}_{j} - 2 {z}_{k} + {z}_{l} )}^{2} ,$$

where l=k+1=j+2. It will be appreciated that the distances L between sequential curve points may vary, for example Lⱼₖ≠Lₖₗ. This would require further generalization of the energy formula, which considers the different lengths,

$$\frac{{L}_{jk}}{{L}_{kl}} .$$

It should be noted that using Euler angles means that any set αᵢ, βᵢ, γᵢ represents a valid orientation which does not need to be normalized, constrained or anything like that. This is not true for quaternions, which have the constraint

$${q}_{i 0}^{2} + {q}_{i 1}^{2} + {q}_{i 2}^{2} + {q}_{i 3}^{2} = 1 ,$$

and may make the optimization numerically more complicated and perhaps require further energy functions.

In some embodiments, a segmental energy function is a 2^{nd} order smoothness energy function, striving to minimize the change in curvature along a sequence of adjacent points. For instance, such energy may be written as

$${E}_{jkl}^{dR} = \frac{{W}_{jkl}^{dR}}{2} ( {R}_{j}^{T} {R}_{k} - {R}_{k}^{T} {R}_{l} ) ,$$

where Rᵢ(qᵢ) is the rotation matrix of the ith sensor. A closely related form which directly uses the quaternion degrees of freedom is

$${E}_{jkl}^{dR} = \frac{{W}_{jkl}^{dR}}{2} ( {q}_{j}^{- 1} {q}_{k} - {q}_{k}^{- 1} {q}_{l} ) .$$

Usually, l=k+1=j+2, and

$${W}_{jkl}^{dR}$$

may be dependent on, for example, lᵢⱼ and lⱼₖ.

In some embodiments, a temporal energy function is used to incorporate temporal sensor data, striving to minimize the deviation between the solved sensor's motion over time and an applied motion model. For example, knowing the current state of the curve and its previous one, denoted by {rᵢ, qᵢ}(t) and {rᵢ, qᵢ}(t−Δt₀) respectively, a simple model for the positions of the next curve is

$${\vec{r}}_{j} ( t + Δ {t}_{1} ) = {\vec{r}}_{i} ( t ) + \frac{Δ {t}_{1}}{Δ {t}_{0}} [ {\vec{r}}_{i} ( t ) - {\vec{r}}_{i} ( t - Δ {t}_{0} ) ] .$$

Therefore, a motion energy which makes sure that the curve does not deviate from the motion model is

$${E}_{i} ( t + Δ {t}_{1} ) = \frac{{W}_{i}^{t}}{2} {\{ {\vec{r}}_{i} ( t ) + \frac{Δ {t}_{1}}{Δ {t}_{0}} [ {\vec{r}}_{i} ( t ) - {r}_{i} ( t - Δ {t}_{0} ) ] - {r}_{i} ( t + Δ {t}_{1} ) \}}^{2} ,$$

and a similar one can be analogously written for the orientations, using qᵢ.

Similarly, a point's linear velocity and angular velocity can be denoted by (vᵢ, ωᵢ) and the motion energy function can then be written as:

$${E}_{i} ( t + Δ {t}_{1} ) = \frac{{W}_{i}^{t}}{2} {\{ {\vec{r}}_{i} ( t ) + Δ {t}_{1} \vec{{v}_{ι}} ( t ) - {\vec{r}}_{i} ( t + Δ {t}_{1} ) \}}^{2}$$

and a similar one can be analogously written for the orientations, using ωᵢ.

In some embodiments, (vᵢ, ωᵢ) can be estimated numerically using consecutive solutions of (rᵢ, qᵢ). In some embodiments, when reliable sensor readings are provided (for example, when the shape sensor is close to the transmitter), then the motion energy weight will be decreased, and the shape solution will be based more on the measured magnetic field values. In some embodiments, however, when the sensor readings are less reliable (for example, when the shape sensor is high above the transmitter), then the motion energy weight will be increased and the shape solution will be based more on the motion model, which will decrease the jitter of the solved shape. In some embodiments, the motion energy then introduces dynamic filtering over time, for jitter reduction, which depends on the reliability of the sensor readings (i.e., on magnetic field strength relative to sensor measurement noise) and which respect all the other constraints in a unified energy function framework.

In some embodiments, the reliability of sensor reading can be estimated by a covariance matrix which is computed using each sensor's measurement noise. For example, some DC magnetometer sensors may provide magnetic measurements with measurement noise of 1 uT (1 micro-tesla), or 0.5 uT (0.5 micro-tesla) or any other noise level. In some embodiments, this noise level can be used when estimating a covariance matrix for the sensor's measurements. Additionally or alternatively, sensor's covariance matrix can be estimated numerically by recording a plurality of sensor measurements at a static position, for example, in a factory calibration process. In some embodiments, the covariance matrix can then be used alongside the sensor readings to compute the sensor's SNR. In some embodiments, when the sensor gets farther away from the transmitter, the sensor's readings of the generated magnetic fields will decrease, and the noise will become more dominant (signal-noise-ratio will decrease). In some embodiments, as the sensor gets closer to the transmitter, the sensor's readings of the magnetic fields will increase, and the noise will become less dominant (signal-noise-ratio will increase). In some embodiments, the signal-noise-ratio (SNR) can then be used to compute the weight for the motion energy, decreasing it as the SNR improves to provide a more rapid curve solution (lower latency) and increasing it as the SNR deteriorates, to provide a smoother curve solution (but with higher latency).

In some embodiments, in the motion energy functions above, rᵢ qᵢ, vᵢ are in transmitter coordinate system. In some cases, it may be beneficial to describe the motion of the device in relative coordinate system. For example, to describe the motion of a point in its preceding point's coordinate system. For example, in some applications, the device may not change in shape, or may change very slowly in shape, in which case the relative motion model can assume vᵢ=0 where vᵢ is the relative linear velocity of point i relative to its preceding point i−1 (in its preceding point's coordinate system). In this case, the energy would strive to keep the device's shape unchanged over time, while the device is not constrained to move in its entirety.

In some embodiments, vᵢ can be used as before but be constrained such that only small relative velocities would be permitted (for example, a device can change in shape with relative velocity of 30 mm per second, or 50 mm per second, or 100 mm per second). Constraining the device's relative motion may further reduce the jitter of the solved curve.

In some embodiments, as mentioned above, by using an energy function which incorporates numerous (or at least one) constraints to solve for the sensor's shape, the solved shape is not only more immune to electromagnetic distortion, but also contains less noise (due to magnetic measurement noise) such that the solved shape is less jittery over time. In some embodiments, this is achieved because the solved shape is constrained (for example, deliberately configured) to mechanically plausible solutions, which reduces the degrees of freedom of the solution. It may also be achieved by combining a motion energy function.

In some embodiments, as indicated in block 508, controller/processor 102 calculates a full localized curve along the tracked portion of the elongated device 110, for example relative to the one or more transmitters 104. For example, the energy function E is minimized, for example, by finding optimal positions and orientations of curve points 202 and 204, so as to minimize the errors with respect to the various constraints incorporated in the function.

In some embodiments, the steps described in blocks 504, 506 and 508 are repeated iteratively.

It will be appreciated that, in some embodiments, for example in case the sensor points 202 are sufficiently close to each other, the calculations described herein are performed based on sensor points 202 with no in-between addition of curve points 204.

In some embodiments, a mechanical constraint relates to known distances along the elongated device 110 between adjacent curve points 202 and/or 204, and/or a known length of a tracked portion of the elongated device 110. In some embodiments, the addition of a sufficient number of curve points 204 between sensor points 202 enables, for example, a linear approximation of the total curve length between two curve points, and/or construction of an energy function E^{length} that incorporates the length constraint, relating to the known distance along the elongated device 110 between two curve points. For example, the energy function Eⱼₖ^{length} relating to the length along the elongated device 110 between two adjacent curve points j and k, may be proportional to the squared difference between the known distance lⱼₖ and the linearly approximated distance according to the momentarily calculated positions \vec{rⱼ} and \vec{rₖ}. In some embodiments, the addition of a sufficient number of curve points 204 between sensor points 202 is also potentially generally beneficial for the accurate representation of a mechanical plausible solved curve with a limited number of points, to accurately model the length constraints as well as other smoothness and/or twist constraints between curve points with sufficient resolution along the curve's length.

In some embodiments, a mechanical constraint relates to a constrained (possibly, softly constrained) orientation difference between adjacent curve points 202 and/or 204. For example, this may be expressed by an energy function

$${E}_{jkl}^{smooth} ,$$

that grows in accordance with the relevant twist orientation and/or derivative differences between adjacent curve points 202 and/or 204.

In some embodiments, a mechanical constraint relates to smoothness of the curve of a tracked portion of the elongated device 110. A corresponding energy function

$${E}_{track} ( \{ {r}_{i} , {q}_{i} \} ) = \frac{1}{2} {\sum }_{i = 1}^{N} {W}_{i} \cdot {‖ {R ( {q}_{i} )}^{T} \cdot {B}_{full} ( {\vec{r}}_{i} , \vec{Θ} ) - {X}_{i} ‖}^{2} .$$

may strive (or be configured to) to minimize the curvature along a sequence of three adjacent curve points j, k and l.

In some embodiments, the curve fitting energy is further generalized by introducing a distortion model with additional parameters. In some embodiments, instead of ignoring sensors 108 which experience high levels of distortion (for example, by decreasing Wis according to distortion levels, as discussed above), information from distorted sensors can be used to model the distortion field. For example, when introducing a surgical tool to a working channel of a curve/shape tracked endoscope, the distorter (i.e., the tool) can be modeled using a set of parameters, for instance: the distortion model parameters constitute tool position inside the working channel and distortion gain of the tool's tip. In some embodiments, the tool's tip can be modeled for instance as a ferromagnetic metal, magnetized by the deliberately generated EM fields. In some embodiments, it can then be viewed as a secondary EM source located at the tool tip's position along the working channel, generating EM fields according to those which exist at its location and with some unknown gain parameter. In any such case or another, the distortion fields can be modeled with a distortion function D(\vec{r}, \vec{Θ}) where r is the position in which the distortion fields are to be computed and \vec{Θ} is a vector of parameters which characterize the distorter (and may also contain other parameters of the optimization). For example, in the case of a working channel tool, \vec{Θ} consists of the position of the tool relative to the tracked endoscope and its distortion gains. In some embodiments, \vec{Θ} consists of the parameters which are unknown and need to be searched for the distortion model to be solved and D(r, \vec{Θ}) is then able to predict the distortion fields at position \vec{r}. E_{track} can then be modified as to account for the predicted distortion field based on the solved distortion model, as follows:

$${E}_{jk}^{twist}$$

The calibrated theoretical fields B(rᵢ) are now replaced with B_{full}(rᵢ) which include the modeled distortion fields:

$${B}_{full} ( {\vec{r}}_{i} ) = B ( {\vec{r}}_{i} ) + \tilde{D} ( {\vec{r}}_{i} , \vec{Θ} ) .$$

{rᵢ}, {qᵢ} now become correlated even without the imposed smoothness and length constraints, just via the shared distortion fields that a plurality of the corresponding sensing elements 108 may sense (each at a different level) at their positions and orientations. In some embodiments, when a source of distortion exists in the device's environment, for example when tool is introduced into the working channel, this can provide for a much more stable full curve solution. Adding the additional smoothness and length constraints further improves the solution.

In some embodiments, the magnetic field measured by sensor element i may be denoted by:

$${X}_{i} = ( 1 + D ) {R}_{i}^{T} B ( {\vec{r}}_{i} ) .$$

where D is indicative of a local distortion field, for example, such as may be created by an introduced magnetic tool at the proximity of the sensor.

In some embodiments, a method for position and/or curve/shape-tracking of an elongated device 110 may include calibration of the curve/shape sensor 106 with respect to a tool, so as to enable position and/or curve/shape tracking of the elongated device 110 that is immune to distortion by the working channel tool, as well as potentially enable tracking of the tool inside the device's working channel.

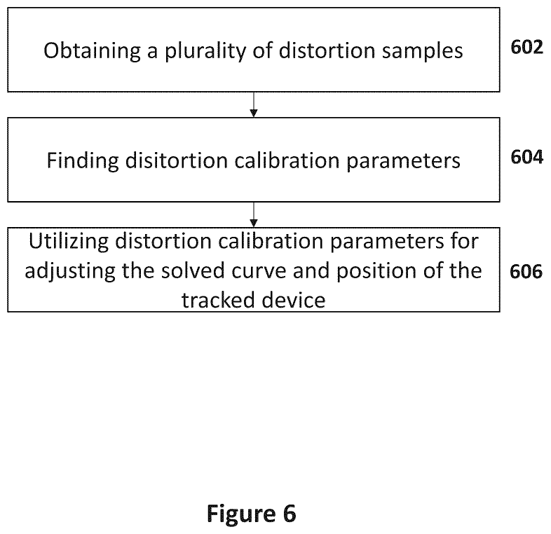

Reference is now made to FIG. 6, showing a flowchart of an exemplary method for working channel tool calibration, according to some embodiments of the invention. In some embodiments, as indicated in block 602, the method includes receiving a large plurality of distortion samples D, while the tool is being inserted into and retracted from a working channel. In some embodiments, the distortion samples are extracted from:

$${X}_{distored} = ( 1 + D ) {X}_{undistorted} .$$

While each distortion matrix D contains nine degrees of freedom (in the case of N_{c}=3 transmitting coils), as indicated in block 604, the method includes finding distortion calibration parameters based on the plurality of distortion samples. For example, controller/processor 102 may reduce D to a solvable number of calibration parameters in D_{reduced}, e.g. reducing the number of distortion possibilities and/or the number of degrees of freedom of the distortion matrix, for example by finding the main possible distortion directions and values, for example by matrix decomposition techniques such as singular value decomposition (SVD) and/or any other suitable techniques. As an example, three of four (N_{D}) constant 3×3 matrices Dᵢ may be found from the calibration, such that a complete distortion matrix may be approximated as:

$$D \approx {\sum }_{i = 1}^{{N}_{D}} {Θ}_{i} {D}_{i} ,$$

$$with$$

$$\frac{‖ D - {\sum }_{i = 1}^{{N}_{D}} {Θ}_{i} {D}_{i} ‖}{‖ D ‖} < 0.05$$

$$or$$

$$\frac{‖ D - {\sum }_{i = 1}^{{N}_{D}} {Θ}_{i} {D}_{i} ‖}{‖ D ‖} < 0.02 ,$$

etc., where ∥x∥ is some norm, for example

$$‖ x ‖ ≡ \sqrt{{\sum }_{i} {x}_{i}^{2} .}$$

Then Θᵢ are a “compressed” set of extra variables (less than nine per sensor) which are also solved for in the optimization process.

In some embodiments, as indicated in block 606, the method includes using the reduced distortion matrix as a calibration matrix, to adjust the measurements by sensor elements 108 and/or to incorporate calibration parameters of distortion matrix D_{reduced} in a distortion energy function E_{distortion}, and/or to adjust the solved curve and position of the tracked elongated device 110.

In some embodiments, the energy function E includes distortion energy function E_{distortion}, which is dependent on a calibration parameter matrix. As indicated in block 604, the method includes finding calibration parameters that minimize the overall calibration error along curve/shape sensor 106, through the various positionings of the tool inside the channel.

In some embodiments, Θᵢ is adjusted by the D matrix such that E_{track}({rᵢ, qᵢ}) is reduced through X. However, another energy E_{distortion} may be included in E_{track}({rᵢ, qᵢ}), which decreases the values of Θᵢ to further constrain the degrees of freedom and keep Θᵢ from “improving” E_{track} at any cost. The use of Θᵢ may be especially beneficial in cases of a strong distortion, so that E_{distortion} limits the growth of θᵢ, for example:

$${E}_{distortion} = \frac{1}{2} {\sum }_{i}^{{N}_{D}} {W}_{{Θ}_{i}} {Θ}_{i}^{2} ,$$

$$or$$

$${E}_{distortion} = \frac{1}{2} {\sum }_{i}^{{N}_{D}} {W}_{{Θ}_{i}} {Θ}_{i}^{4} ,$$

W_{Θ}_{ᵢ} may be learned as part of the calibration described above.

In some embodiments, during operation, when the tool is introduced into a channel, a location of a tip of tool can be found by comparing a theoretical distortion function to calculated distortion values sensed by the various sensor elements 108, for example according to the reduced distortion matrix D_{reduced}.

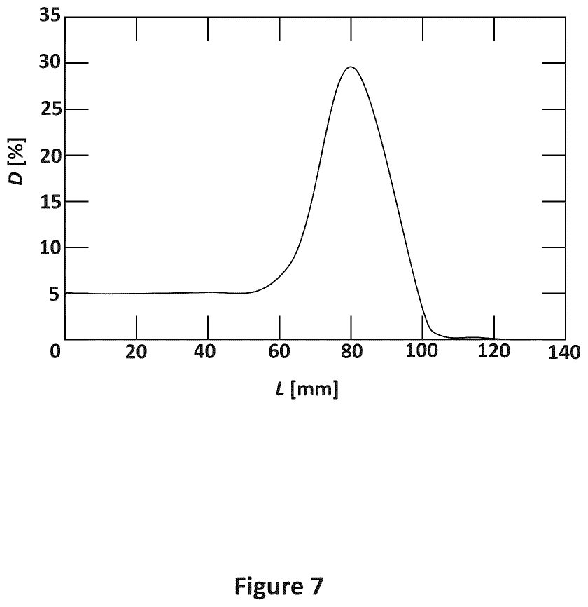

For example, as shown for example in FIG. 7, a theoretical diagram of the amount of distortion sensed along the elongated device 110, according to some embodiments of the invention. For example, a tip of tool may cause a large distortion, with a weaker distortion tail generated, for example, by the tool shaft (also referred to as tool body) behind the tip. The theoretical diagram may be compared and fitted to a series of distortion values calculated for each sensor element 108. For example, a distortion value for a certain sensor element may be calculated by a rooted mean square (RMS) of the reduced distortion matrix. Accordingly, the location of the tool tip along the elongated device 110 may be monitored.

In some embodiments, in the calibration process, another method to obtain both tool immunity and especially better tool tracking is to save the whole data, Nₓ D matrices for each position of the tool along the receiver. In some embodiments, this reduces the degrees of freedom to just a single one—the position of the tool inside the working channel.

It should be noted that there are several ways in which the distortion fields can be modeled, depending on the kind of distorter. For example, in some embodiments, as discussed above, tool distortion can be modeled by parametrizing its position along the tracked length of the endoscope and its distortion gains using as few as two parameters (position+single gain). In some embodiments, its corresponding distortion fields can then be modeled as dipole fields generated at the tool's tip. In some embodiments, in case of a tool with a prolonged magnetic body (not just a tip), a series of dipoles can be used to model the fields generated by the tool. In some embodiments, the system may be aware of the type of inserted tool. For example, the user may indicate to the system what kind of tool it is being introduced. For example, in one embodiment, the system may auto-detect the presence of a tool inside the working channel by sensing the distortion level of its sensor elements and/or by using a magnetic sensor (not even a position sensor) near the entrance to the working channel, for example inside the device's handle. In some embodiments, the system may then automatically prompt the user with a list of possible tools to select from. Alternatively or additionally, the system may auto-detect the type of tool introduced into the working channel by analyzing the distorted fields sensed by the sensor elements 108. In some embodiments, the system may then automatically prompt the user with a suggested tool, as was automatically detected, for the user to approve or modify. In some embodiments, knowing which tool exactly is introduced into the working channel, the system may load a pre-calibrated distortion model of the inserted tool. For example, the system may know that a tool consists of a magnetic tip and a non-magnetic body. The system may further know that the magnetic tip is 1 mm long and may also have information of the exact distortion gain that the tip generates in its interaction with the EM generated fields. The system may then only need to search for the tool position inside the tracked endoscope, in which case the distortion parametric vector Θ reduces to a single scalar, which may improve the accuracy of the final solved curve.

In some embodiments, one or more sensor elements are fitted to a distorter, such as a tool, to aid in the detection, modeling and compensation of the tool distortion. In some embodiments, the sensor elements 108 are calibrated on the tool such that the tool distortion is static relative to the sensors and compensated in calibration. In some embodiments, the tool's position and orientation can then be tracked, and its position and orientation can be shared in the general energy-based optimization process to reduce the number of parameters in the optimization. In some embodiments, similarly, the position and orientation of a distorter (such as a robotic arm or such as a tool) may be provided additionally or alternatively by an electronic or mechanical control system such as a robotic drive system. In some embodiments, alternatively, magnetic field measurements from the tool's one or more sensors can be plugged into the general energy-based optimization framework and provide more information for the optimization process, to improve the convergence process.

In some embodiments, another type of distortion can be modeled under the same framework described above. In the case of a global dynamic EM distortion (as opposed to local EM distortion), for example, in the case of a metallic C-arm which is located above the EM transmitter and in proximity to the EM curve/shape sensor (e.g., EM curve/shape sensed endoscope), the C-arm may create distortion fields which are global in the sense that they vary slowly in space (since the distortion source is rather far from the EM sensor elements). In this case the distortion can be modeled for example as a ferromagnetic or diamagnetic ball or sphere located at some unknown 3D position in space. In some embodiments, the distortion shape/object can then be modeled as a secondary source, generating fields which are proportional to those fields deliberately generated at its position, and with unknown gains. In some embodiments, Θ can then consist of a 3D position and one or more gains which need to be solved. Plugging this into E_{track} as described above, all the sensor elements are solved under the same global distorter and in addition the distorter's position can be solved. This can be used for example to detect the presence or absence of a C-arm in the proximity of the transmitter, which may have several applicative uses.

In some embodiments, multiple sensors or sensor arrays can be localized together under the same energy minimization framework. In some embodiments, solving multiple EM curve/shape sensors simultaneously has the potential advantage of sharing distortion information (mostly global distortion) among multiple EM sensors. For example, in the case of global distortion, such as that caused by a C-arm, all sensors in space experience the distortion caused by the same distorter, so that the parametrization of the distorter (e.g., its position and gains in space) can be shared among all sensors. In this case, the energy function E can be generalized as such:

$$E ( {r}_{i}^{j} , {q}_{i}^{j} ) = {\sum }_{j = 1}^{M} {E}_{j} ( {r}_{i}^{j} , {q}_{i}^{j} )$$

Here M is the number of tracked devices, and each Eⱼ is the energy function of specific tracked device j. The j-th device may be a full EM curve/shape sensor, having both E_{track} as well as imposed constraints Eₛₘₒₒₜₕ, E_{length} as described above, but it can also be a single tracked EM sensor, such as a reference sensor attached to the patient's body, or a full set of such reference sensors, attached for example in a grid. In some embodiments, in the case of a full grid of sensors, additional or different constraints may be imposed on the solved sensor grid, such as structural energy, E_{struct} rather than Eₛₘₒₒₜₕ or E_{length}. In some embodiments, the framework described above is flexible enough to accommodate for any kind of imposed constraint on the solved positions and orientations of the tracked one or more devices. In some embodiments, the potential advantage of adding reference sensors into the mix is again the ability to share the information about distortion between different tracked sensors, especially in the case of global distortion. For example, in the case of global distortion caused by a C-arm, reference sensors placed on the patient's chest may sense the distortion and their measurements add a lot of information when solving for the global distortion's model parameters Θ in the combined energy function. This not only improves the solution of Θ in the optimization but more importantly improves the accuracy of the solved sensors, which are for example used to provide full curve/shape sensing of an endoscope. In some embodiments of the present disclosure, such sensors grid, being attached to a patient's chest, may also be used to track a patient's motion during procedure. It can also be used to track a patient's breathing. It can also be used to model the patient's body motion and deformation which can be used to model the deformation of a patient's internal organs.

In some embodiments of the present disclosure, instead of describing just a single model for distortion, several models can be combined in a single or multiple D(r, Θⱼ) s. For example, each fully tracked endoscope may model its own Dⱼ(r, Θⱼ) to accommodate for tool interference in its working channel while all tracked sensors (including reference sensors) may share a single D_{global}(r, Θ_{global}) to model the global distortion which may be caused for example by a C-arm.

In some embodiments, each position and/or curve/shape tracked device itself (or even a non-tracked device or tool) may cause distortion on other tracked devices. For example, in the case of two position and/or curve/shape tracked endoscopes, each endoscope may contain ferromagnetic materials which react to the generated fields and create local distortion fields at its proximity. In the case of multiple tracked devices, the devices may create mutual distortion on each other. For example, when two position and/or shape tracked endoscopes are touching (or are at close proximity), the position and/or tracked curve/shape of each endoscope may be slightly distorted at the point of contact (or close proximity). In the general energy-based optimization framework, since E_{track} is aware of distortion levels, it may reduce the corresponding weight for the distorted sensors along the tracked curves. The location of these sensors would then be interpolated or extrapolated using neighboring sensors through Eₛₘₒₒₜₕ, E_{length}.

Alternatively, in another embodiment, the local distortion caused by each tracked device can be modeled and used directly in the optimization process. Since the curve/shape of each device is solved (and described through rᵢ, qᵢ), this may serve, along with other additional parameters (such as distortion gains) in the distortion parameters vector Θ as a distortion model of each tracked device. This model, along with its predicted distortion fields, can be used in the optimization process to solve the accurate shapes of the two devices in proximity, without decreasing the distorted sensors' weights, as described above. In some embodiments, the mutual distortion between tracked devices (or the one-sided distortion which may be caused by a distorter on a tracked device) can be detected and utilized for various purposes, for example, alerting the user in application on the presence of distorter.

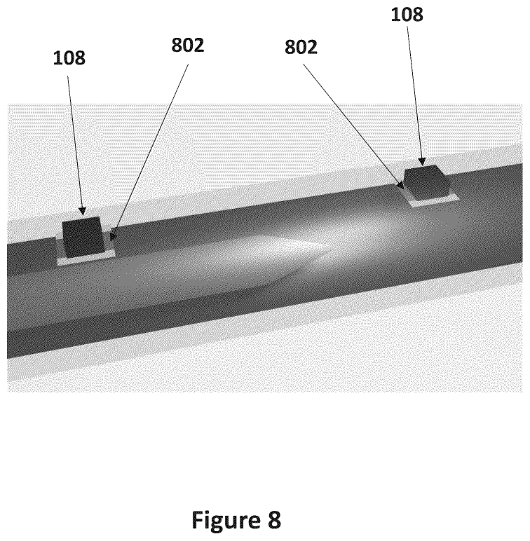

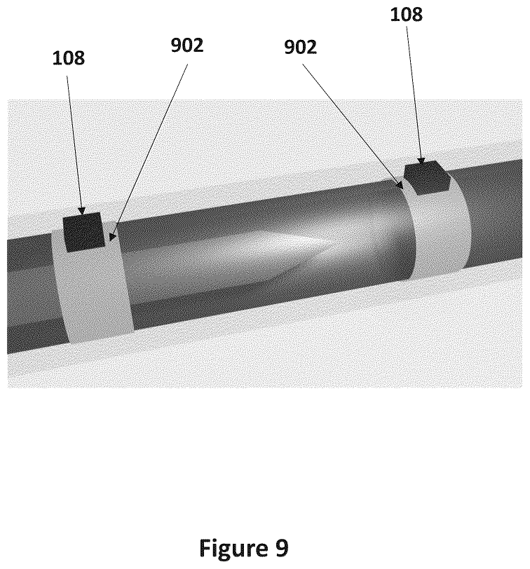

In some embodiments, local EM distortion caused by magnetic tools in an endoscope's working channel can be reduced using magnetic shielding. In some embodiments, in this case, materials of high magnetic permeability are deliberately placed between each EM sensor element and the potential position of a distortion source, to shield it from potential distortion. For example, in the case of EM tracked endoscope, the endoscope's working channel can be made of or wrapped with high permeability magnetic materials, such as permalloy, supermalloy, MuMetal, or even ferritic stainless-steel (such as 17-4), or any other suitable material. These materials may serve as “magnetic conductors”, so that they conduct the magnetic field lines, to shield the fields created by local distorters from the nearby EM sensor elements. In some embodiments, digital magnetometers can be assembled on a Flexible Printed Circuit (FPC) and wrapped around the working channel of an endoscope. In some embodiments, in order to provide shielding from magnetic tools introduced inside the working channel, thin Permalloy films (pads) 802 can be placed below the sensors 108 such that they shield between the discrete EM sensors and the working channel, as depicted for example in FIG. 8. In some embodiments, the sensors would then experience a reduced distortion fields due to the introduced tools. In addition, the sensors 108 may sense slightly modified fields of the deliberately generated EM fields by the EM transmitter. In some embodiments, to solve that, the sensors can be calibrated with the magnetic shielding to balance this effect. In some embodiments, the magnetic shield film placed below each sensor may also serve as a local mechanical stiffener for the sensor on the FPC. In some embodiments, the local mechanical stiffener is important for cases where the FPC is wrapped around the working channel, for example in a helix manner. In some embodiments, with the local mechanical stiffener, the chance of breaking one of the sensor's pads is much reduced. In some embodiments, the magnetic shield film may therefore serve as both shield from undesired local EM distortion fields (such as those created by tools introduced into the endoscope's working channel) as well as increase the robustness of the FPC assembly and integration inside the endoscope. In some embodiments, instead of using magnetic shield pads, a tube 902 can be used to shield the sensor 108 from tool distortion. In some embodiments, the shielding tube 902 can be placed below the sensor 108 and wrap the working channel, as depicted for example in FIG. 9.

In some embodiments, using low-frequency EM fields may reduce the distortion caused by eddy currents. In some embodiments, in a low-frequency EM tracking system, the generated fields are of low frequency (for example, slower than 300 Hz). In such a system, the sensor can consist for example of DC magnetometers, which unlike sensing coils which are based on Faraday's law of induction, their Signal-to-Noise Ratio (SNR) is unaffected by the transmission frequency. In this case, reducing the frequency of the transmitted fields reduces the eddy currents in nearby conductors, without affecting the SNR of the sensors. In some embodiments, this reduces the overall distortion while maintaining the same SNR.

In some embodiments, the one or more transmitters 104 generate EM fields of a special multi-frequency profile. For example, the transmitter generates rectangular, triangular or “chainsaw” fields. In some embodiments, these fields consist of multiple frequencies, as opposed to standard EM fields which are usually just sinusoidal (and thus each consists of a single frequency). In some embodiments, a distorter, reacting to the generated fields, may respond differently to each different frequencies, according to some spectrum profile which depend on its material and geometry. In some embodiments, by analyzing the sensed profile, the receiver may then detect a distorter, as well as model the distortion and compensate for the distortion, according to a distortion detected in the transmitted profile. In some embodiments, such analysis may require detailed analysis of the sensed signal using extensive computational effort. However, in some embodiments, since the transmitted fields may be of low base frequency (for example, lower than 300 Hz) the receiver can fully analyze the raw sensed signals in real-time. For example, if the EM transmitter generates a set of square waves at different frequencies, the EM receiver is expected to sense a superposition of these square waves. However, with a distorter present, the square waves may be deformed (due to the distorter) and a deformed square wave may then be sensed by the EM sensors, which may aid in the detection and compensation of EM distortion and distorters.

In some embodiments, the one or more transmitters 104 generate EM fields of a special multi-frequency profile. For example, the transmitter generates EM fields consisting of multiple harmonies of the base frequency f₀. For example, the transmitter may transmit EM fields such as:

$$cos ( 2 π {f}_{0} t ) - \frac{1}{4} sin ( 4 π {f}_{0} t ) or sin ( 2 π {f}_{0} ) + \frac{1}{8} sin ( 6 π {f}_{0} )$$

or any other combination of a base frequency and weighted harmonies of different strengths. In some embodiments, having knowledge of the transmitted frequencies (and harmonies), the processor in the controller 102 can then analyze the sensed fields and compare them with the expected frequency profile (including the harmonics). For example, in the case of transmitting 1X base frequency f₀, and

$$\frac{1}{8} X$$

harmony 3f₀, It is expected to pickup 1X and

$$\frac{1}{8} X$$

of the frequency and the harmony on the receiver. But if the ratio is not 1:⅛ then it means that a distorter is present and that it reacted differently to f₀ and the harmony 3f₀. By assuming a pure eddy-current distortion, it is expected that the distortion will be 3 times stronger for the 3f₀ harmony. The processor in the controller 102 can then apply a formula to extract the distortion field from the DFT of the base frequency and the harmony, by assuming for example that X_{f0}=B+E, X_{3f}_{₀}=B+3E, where B is the undistorted field and E is the distortion field, then B=3X_{f0}−X_{3f}_{₀}. Using this or a similar formula, multi-frequency measurements can be used to detect and extract the distortion field, when the field generator generates multi-frequency fields (including harmonies) for one or more of its transmitting coils.

In some embodiments, further to EM curve/shape sensing, combining additional sensor types may be helpful in detecting and measuring distortion. For example, a pressure sensor may be used to detect passage of a tool inside an endoscope, to inform of presence of distortion source. Subsequent measurement of an adjacent tracking sensor may be interpreted as the amount of distortion. Additional examples of sensor types may be axial tension, impendence, thermometer, IMU, etc.

In some embodiments, while the example of an endoscope with a working channel was used herein, it should be appreciated that any other tracked device can be used instead and the methods of distortion detection, modeling and compensation are equivalently applicable to that general tracked device.

In addition, while the example of clinical tools introduced into an endoscope's working channel was used throughout the disclosure, it should be appreciated that the same methods of detection, modeling and compensation are equivalently applicable to any kind of distorter (local or global, as explained above), whether introduced through a working channel or externally to the tracked device, such as perpendicularly to the tracked device, or in proximity with the tracked device, or in any other way which distorts the tracked device.

In some embodiments, while the example of specific energy functions was used herein, it should be appreciated that any energy function of similar nature, which describe mechanical or magnetic or tracking or motion characteristics of the tracked device can be used additionally or alternatively to the energy functions described herein, and the methods of solving the device's curve using a unified energy function are equivalently applicable to any other energy functions.

As used herein with reference to quantity or value, the term “about” means “within ±20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A system for position and/or curve/shape-tracking of an elongated device, comprising:
a. a curve/shape sensor comprising a plurality of sensor elements positioned on said elongated device;
b. one or more transmitters;
c. a controller comprising a processor; said processor comprising instructions for:
i. obtaining a plurality of points along a tracked portion of said elongated device;
ii. allocating, for each point from said plurality of points, a local energy function dependent on an estimated position and orientation of said tracked portion of said elongated device;
iii. generating a resultant unified energy function for a full shape and a position of an entire tracked portion of said elongated device;
iv. calculating a fully localized curve along said tracked portion of said elongated device by minimizing said unified energy function.

2. The system according to claim 1, wherein said local energy function incorporates relevant mechanical and sensor measurement constraints for said each point; and
wherein said unified energy function is constructed based on said allocated local energy functions and segmental energy functions that relate to said constraints of mechanical properties of said elongated device, with respect to relative locations and orientation of adjacent plurality of the predetermined points.

3. (canceled)

4. The system according to claim 1, wherein said fully localized curve is calculated relative to said one or more transmitters.

5. The system according to claim 1, wherein said plurality of points are one or more of:
a. a predetermined plurality of points;
b. a sensor point, which is a point in which said sensor elements of said shape sensor are located; and
c. a curve point, which is a virtual predetermined point positioned at predetermined intervals between sensor points.

6. (canceled)

7. The system according to claim 5, wherein there are one or more of:
a. a plurality of sensor points along said tracked portion of said elongated device; and
b. one or more curve points between sensor points.

8. (canceled)

9. The system according to claim 1, wherein said local energy function incorporates constraints related to a sensed magnetic field.

10. The system according to claim 1, wherein the processor further comprises instructions for allocating a weight for each local energy function, based on a certainty value, related to a certainty that a measurement is accurate.

11. The system according to claim 2, wherein said segmental energy function is characterized by being one or more of:
a. a length energy function, related to known distances along said elongated device between adjacent points from said plurality of points or a known length of said tracked portion of said elongated device;
b. an orientation energy function, related to a limited possible orientation difference between adjacent points from said plurality of points;
c. at least one selected from the group consisting of:
i. at least one energy function corresponding to the tracking approximation of a sensor point;
ii. at least one energy function corresponding to the length approximation between adjacent points;
iii. at least one energy function corresponding to the distortion approximation of a sensor point;
iv. at least one energy function corresponding to the orientation difference between adjacent points; wherein said orientation energy function grows in accordance with said orientation difference between said adjacent points;
v. at least one energy function corresponds to the twist difference between adjacent points;
vi. at least one energy function corresponds to the smoothness/curvature difference between adjacent points; and
vii, wherein at least one energy function corresponds to the motion difference between a point and a motion model of that point;
d. a smoothness energy function configured for minimizing a curvature along a sequence of adjacent points; and
e. a motion energy function configured for minimizing a jitter of said calculated fully localized curve.

12. The system according to claim 11, wherein said length energy function is characterized by one or more of:
a. incorporating an approximation of a total curve length between two points, and a known distance along said elongated device between said two points;
b. relating to a length along said elongated device between two adjacent curve points, and is proportional to the squared difference between a known distance and a linearly approximated distance according to momentarily calculated positions.

13-18. (canceled)

19. The system according to claim 1, wherein said processor further comprises instructions for calibrating said curve/shape sensor with respect to a magnetic field distortion imposed by a tool inserted into said elongated device; and
wherein said calibration includes one or more of:
a. obtaining a plurality of distortion samples;
b. finding distortion calibration parameters by reducing a distortion matrix based on said distortion samples;
c. using said distortion calibration parameters to adjust said calculated fully localized curve of said tracked portion of said elongated device;
d. monitoring a location of a tool tip; wherein said monitoring comprises comparing and fitting a theoretical diagram of a distortion distribution to a series of distortion values calculated for a plurality of points along said tracked portion of said elongated device; and wherein said distortion value for a certain sensor element is calculated by a rooted mean square of said reduced distortion matrix.

20-23. (canceled)

24. The system according to claim 1, wherein said one or more transmitters are configured for transmitting one or more multi-frequency EM fields comprising one or more harmonies of a base frequency; and wherein said processor comprises further instructions for:
a. analyzing sensed magnetic fields;
b. comparing said sensed magnetic fields with an expected frequency profile;
c. extracting a distortion field; and
d. utilizing said distortion field in said calculating.

25. (canceled)

26. A computer implemented method for position and/or curve/shape-tracking of an elongated device performed by a curve/shape sensor comprising a plurality of sensor elements positioned on said elongated device, the method comprising:
a. obtaining a plurality of points along a tracked portion of said elongated device; said plurality of points corresponding to readings from said plurality of sensor elements;
b. allocating, for each point from said plurality of points, a local energy function dependent on an estimated position and orientation of said tracked portion of said elongated device;
c. generating a resultant unified energy function for a full shape and a position of an entire tracked portion of said elongated device;
d. calculating a fully localized curve along said tracked portion of said elongated device by minimizing said unified energy function.

27. The computer implemented method according to claim 26, wherein said local energy function incorporates relevant mechanical and sensor measurement constraints for said each point; and wherein said unified energy function is constructed based on said allocated local energy functions and segmental energy functions that relate to said constraints of mechanical properties of said elongated device, with respect to relative locations and orientation of adjacent plurality of the predetermined points.

28. (canceled)

29. The computer implemented method according to claim 26, wherein said fully localized curve is calculated relative to one or more transmitters.

30. The computer implemented method according to claim 26, wherein said plurality of points are one or more of:
a. a predetermined plurality of points;
b. a sensor point, which is a point in which said sensor elements of said shape sensor are located; and
c. a curve point, which is a virtual predetermined point positioned at predetermined intervals between sensor points; and
wherein there are a plurality of sensor points along said tracked portion of said elongated device;
wherein there are one or more curve points between sensor points.

31-33. (canceled)

34. The computer implemented method according to claim 26, wherein said local energy function incorporates constraints related to a sensed magnetic field.

35. The computer implemented method according to claim 26, further comprising allocating a weight for each local energy function, based on a certainty value, related to a certainty that a measurement is accurate.

36. The computer implemented method according to claim 27, wherein said segmental energy function is a length energy function, related to known distances along said elongated device between adjacent points from said plurality of points or a known length of said tracked portion of said elongated device;
wherein said length energy function incorporates a linear approximation of a total curve length between two points, and a known distance along said elongated device between said two points;
wherein said length energy function relates to a length along said elongated device between two adjacent curve points, and is proportional to the squared difference between a known distance and a linearly approximated distance according to momentarily calculated positions.

37-38. (canceled)

39. The computer implemented method according to claim 27, wherein said segmental energy function is characterized by being one or more of:
a. an orientation energy function, related to a limited possible orientation difference between adjacent points from said plurality of points;
b. at least one selected from the group consisting of:
i. at least one energy function corresponding to the tracking approximation of a sensor point;
ii. at least one energy function corresponding to the length approximation between adjacent points;
iii. at least one energy function corresponding to the distortion approximation of a sensor point;
iv. at least one energy function corresponding to the orientation difference between adjacent points; wherein said orientation energy function grows in accordance with said orientation difference between said adjacent points;
v. at least one energy function corresponds to the twist difference between adjacent points;
vi. at least one energy function corresponds to the smoothness/curvature difference between adjacent points; and
vii, wherein at least one energy function corresponds to the motion difference between a point and a motion model of that point;
c. a smoothness energy function configured for minimizing a curvature along a sequence of adjacent points; and
d. a motion energy function configured for minimizing a jitter of said calculated fully localized curve.

40-43. (canceled)

44. The computer implemented method according to claim 26, further comprising calibrating said curve/shape sensor with respect to a magnetic field distortion imposed by a tool inserted into said elongated device; and
wherein said calibrating includes one or more of:
a. obtaining a plurality of distortion samples;
b. finding distortion calibration parameters by reducing a distortion matrix based on said distortion samples; wherein said distortion value for a certain sensor element is calculated by a rooted mean square of said reduced distortion matrix; and
c. using said distortion calibration parameters to adjust said calculated fully localized curve of said tracked portion of said elongated device;
d. monitoring a location of a tool tip; wherein said monitoring comprises comparing and fitting a theoretical diagram of a distortion distribution to a series of distortion values calculated for a plurality of points along said tracked portion of said elongated device.

45-49. (canceled)
